(19) United States
(12) Patent Application Publication
(10) Pub. No.: US 2026/0232790 A1
(43) Pub. Date: Aug. 13, 2026

(54) ADJUVANT FORMULATION INCLUDING LOW VISCOSITY CHITOSAN OR CHITOSAN DERIVATIVES

(71) Applicant: Merck Sharp & Dohme LLC, Rahway, NJ (US)

(72) Inventors: Erica L. Strable, Gilbertsville, PA (US); David S. Thiriot, Harleysville, PA (US); Eman Alkurdi, Huntingdon Valley, PA (US); Colleen M. Barr, Pennsburg, PA (US)

(73) Assignee: Merck Sharp & Dohme LLC, Rahway, NJ (US)

(21) Appl. No.: 19/151,645

(22) PCT Filed: Feb. 5, 2024
(86) PCT No.: PCT/US2024/014372

§ 371 Date: Jul. 29, 2025

Related U.S. Application Data
(60) Provisional application No. 63/483,637, filed on Feb. 7, 2023.

(51) Int. Cl. A61K 39/155; A61K 39/00; A61K 39/39; A61K 47/02; A61K 47/22; A61K 47/26; A61P 31/14; C07K 14/005

(52) U.S. Cl. CPC A61K 39/155; A61K 39/39; A61K 47/02; A61K 47/22; A61K 47/26; A61P 31/14; C07K 14/005; A61K 2039/5258; A61K 2039/55505; A61K 2039/55583; A61K 2039/575; C12N 2710/20034; C12N 2760/18522; C12N 2760/18534

(57) ABSTRACT

The invention relates generally to the use of chitosan and chitosan derivatives as an adjuvant in a vaccine composition. More specifically, the invention relates to pharmaceutical compositions and formulations that include a low viscosity chitosan or a trimethyl chitosan.

### CROSS-REFERENCE TO RELATED APPLICATIONS

This application claims the benefit of priority to U.S. Provisional Application No. 63/483,637, filed Feb. 7, 2023, the disclosure of which is incorporated by reference herein in its entirety.

### FIELD OF THE INVENTION

The invention relates generally to the use of a lower viscosity chitosan or chitosan derivative as an adjuvant in a vaccine composition. In specific embodiments, the invention relates to pharmaceutical compositions and formulations comprising a trimethyl chitosan adjuvant.

### BACKGROUND

Chitosan is a linear polysaccharide derived from the abundant natural product chitin, which has been studied for many potential applications in the fields of food, manufacturing, and medicine, including as an adjuvant for use in vaccines. Commercial sources of chitosan typically have an average molecular weight of about 100 kDa or more. Chitosan that has been extensively digested to small numbers of monomeric units per polymer chain is known as chitosan oligosaccharide. For chitosan solutions, high viscosity and low solubility can pose challenges to successfully using chitosan in vaccine manufacturing processes and formulations. Chitosan is soluble at acidic pH but becomes insoluble at pH above about 6.5. This limits the pH range of potential vaccine formulations. Chitosan solutions can also be quite viscous, which can be challenging during bioprocessing, sterile filtration, formulation, and filling into containers. There exists a need for an adjuvant that retains the desired properties of chitosan, such as vaccine adjuvant activity, while minimizing challenges related to solubility and viscosity.

### SUMMARY OF THE INVENTION

The invention provides a pharmaceutical composition comprising an active biological ingredient (ABI), a chitosan or a chitosan derivative, and a pharmaceutically acceptable carrier.

The invention further provides a pharmaceutical composition made by mixing a vaccine and a chitosan or chitosan derivative; wherein the vaccine comprises an ABI and a pharmaceutically acceptable carrier.

The invention also provides a method of inducing an immune response to an antigen in a human patient comprising administering to the patient a pharmaceutical composition comprising an ABI comprising an antigen, a chitosan or chitosan derivative, and a pharmaceutically acceptable carrier.

The invention also provides a method of inducing an immune response to an antigen in a human patient comprising co-administering to the patient (a) a pharmaceutical composition comprising an ABI comprising an antigen and (b) a chitosan or chitosan derivative.

The invention also provides a kit comprising: (a) a vaccine comprising an active biological ingredient (ABI) comprising an antigen; and (b) a chitosan or a chitosan derivative. In some embodiments, the kit also includes instructions for administering to a human patient the vaccine and the chitosan or chitosan derivative.

In one embodiment, the invention provides a method of inducing a neutralizing immune response against an antigen in a patient in need thereof comprising: administering to the patient a pharmaceutical composition comprising a chitosan or chitosan derivative, an active biological ingredient (ABI) comprising an antigen, and a pharmaceutically acceptable carrier, whereby the administration of the pharmaceutical composition induces a neutralizing immune response against the antigen in the patient.

### Definitions

As used throughout the specification and in the appended claims, the singular forms “a.” “an,” and “the” include the plural reference unless the context clearly dictates otherwise.

As used throughout the specification and appended claims, the following definitions and abbreviations apply:

AAHS: As used herein, the term “AAHS” refers to an amorphous aluminum hydroxyphosphate sulfate adjuvant.

Active Biological Ingredient (ABI) or Active Pharmaceutical Ingredient (API): As used herein, the term ABI refers to an active ingredient of a pharmaceutical formulation that is capable of eliciting an immune response against an infectious agent or pathogen, e.g., a DNA, mRNA, protein, peptide, virus-like particle, attenuated or inactive virus. An ABI is the component of a biological pharmaceutical formulation that is useful for inducing a desired positive therapeutic effect when administered to a patient, e.g. treating or preventing a disease or condition, which may include halting or delaying the progression of a disease or pathological condition, reducing the severity or duration of the clinical symptoms of the disease, prolonging the survival of a patient relative to the expected survival in a similar untreated patient, and inducing complete or partial remission of the disease or condition. In the compositions and methods of the invention, the ABIs comprise antigens and include, e.g., HPV VLPs, RSV proteins, which is a component of the compositions or formulations disclosed herein that is biologically active (e.g. capable of inducing an appropriate immune response) and confers a therapeutic or prophylactic benefit to a person or animal in need thereof or who could benefit therefrom. An “active pharmaceutical ingredient” (or “API”) refers to any active ingredient in a pharmaceutical formulation that is useful for treating or preventing a pathological disease or condition, including but not limited to, antibodies and antigen-binding fragments thereof, proteins, and small molecules. As used herein, the ABI or API may be a vaccine ingredient.

About: As used herein, the term “about,” when used herein in reference to a value, refers to a value that is the same as or, in context, is similar to the referenced value. In general, those skilled in the art, familiar with the context, will appreciate the absolute amount and/or relative degree of difference encompassed by “about” in that context. For example, in some embodiments, the term “about” encompasses a range of values within 25%, 20%. 19%. 18%, 17%, 16%, 15%, 14%, 13%, 12%, 11%, 10%, 9%, 8%, 7%, 6%, 5%, 4, 3%, 2%, 1%, or less of the referenced value.

Acid-Soluble Chitosan: As used herein, the term “acid-soluble chitosan” refers to chitosan that is prepared by solubilizing a chitosan powder in an acid. Examples of acid-soluble chitosans include non-salt or free base forms of chitosan, e.g., Sigma-Aldrich Product Number 448869.

Adjuvant: As used herein, the term “adjuvant” refers to a composition or compound that is capable of enhancing the immune response against an antigen of interest. Adjuvants are substances or combinations of substances that are used in conjunction with a vaccine antigen to enhance (e.g., increase, accelerate, prolong and/or possibly target) a specific immune response to the vaccine antigen or modulate to a different type (e.g., switch a Th1 immune response to a Th2 response, or a humoral response to a cytotoxic T cell response) in order to enhance the clinical effectiveness of the vaccine. In some embodiments, the adjuvant modifies (Th1/Th2) the immune response. In some embodiments, the adjuvant boosts the strength and longevity of the immune response. In some embodiments, the adjuvant broadens the immune response to a concomitantly administered antigen. In some embodiments, the adjuvant is capable of inducing strong antibody and T cell responses. In some embodiments, the adjuvant is capable of increasing the polyclonal ability of the induced antibodies. In some embodiments, the adjuvant is used to decrease the amount of antigen necessary to provoke the desired immune response and provide protection against the disease. In some embodiments, the adjuvant is used to decrease the number of injections needed in a clinical regimen to induce a durable immune response and provide protection against a disease. Adjuvant containing formulations described herein may demonstrate enhancements in humoral and/or cellular immunogenicity of vaccine antigens, for example, subunit vaccine antigens. Adjuvants of the invention are not used to deliver antigens, antibodies, active biological ingredients (ABIs), active pharmaceutical ingredients (APIs), or VLPs.

Administration: As used herein, the term “administration” or “administering” refers to the act of providing an active agent (e.g., ABI), composition, or formulation to a subject. Exemplary routes of administration to the human body include administration through the eyes (ophthalmic), mouth (oral), skin (transdermal), nose (nasal), lungs (inhalant), rectal, vaginal, oral mucosa (buccal), ear, by injection (e.g., intravenously (IV), subcutaneously, intratumorally, intraperitoneally, intramuscularly (IM), intradermally (ID) etc.) and the like.

Agent: As used herein, the term “agent” refers to a particle, compound, molecule, or entity of any chemical class including, for example, a VLP, a small molecule, polypeptide (e.g., a protein), polynucleotide (e.g., a DNA polynucleotide or an RNA polynucleotide), saccharide, lipid, or a combination or complex thereof. In some embodiments, the term “agent” refers to a compound, molecule, or entity that includes a polymer, or a plurality thereof.

Antibody: As used herein, the term “antibody” (or “Ab”) refers to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies

Antigen: As used herein, the term “antigen” refers to any antigen that can generate one or more immune responses. The antigen may be a protein (including recombinant proteins), VLP, polypeptide, or peptide (including synthetic peptides). The antigen may be one that generates a humoral and/or CTL immune response.

Chitosan: As used herein, the term “chitosan” refers to a polysaccharide containing randomly distributed β-(1→4)-linked D-glucosamine (deacetylated unit) and N-acetyl-D-glucosamine (acetylated unit) (i.e., α-(1→4)-2-amino-2-deoxy-β-d-glucan), which is mostly deacetylated). Chitosan may be isolated after chemical modification of crustacean chitin shells or other natural sources like fungi. Alternatively, chitosan may be generated by a chemically synthetic route. Chitosan could be further modified by various degrees of acetylation, alkylation, chain length and the addition of other chemical modifications, such as adding thiols, amines, and other functional groups. As used herein, chitosan refers to a class of molecules having a degree of deacetylation above 75%. The term “chitosan” as used herein includes acid-soluble chitosan and water-soluble chitosan.

Chitosan Adjuvant: As used herein, the term “chitosan adjuvant” refers to a composition comprising a chitosan, a chitosan derivative, or a combination of a chitosan and a chitosan derivative compound that is capable of enhancing the immune response against an antigen of interest.

Chitosan Derivative: As used herein, the term “chitosan derivative” refers to chitosan that was modified by various degrees of alkylation, or other chemical modifications. As used herein, chitosan derivative includes N-quaternary chitosan derivatives. In one embodiment, the chitosan derivative is trimethyl chitosan.

Co-administration: As used herein, the term “co-administration” or “co-administering” in relation to the chitosan adjuvant and a pharmaceutical formulation (e.g., an HPV vaccine) refers to administration of a chitosan adjuvant and a pharmaceutical formulation (e.g., an HPV vaccine) concurrently, i.e., simultaneously in time, or sequentially, i.e., administration of an HPV vaccine followed by administration of the chitosan adjuvant (or vice versa). That is, after administration of the HPV vaccine (or chitosan adjuvant), the chitosan adjuvant (or HPV vaccine) is administered substantially immediately after the HPV vaccine (or chitosan adjuvant) or the chitosan adjuvant (or the HPV vaccine) is administered after a period of time following administration of the HPV vaccine (or chitosan adjuvant): the period of time is, in some embodiments, within 1, 2, 3, 5, 10, 15, 20, 25, 30, 45, or 60 minutes.

Deacetylation: As used herein, the term “deacetylation” refers to the removal of an acetyl group from an organic compound. The deacetylation of solids may be measured via methods known in the art, such as, NMR, UV (EP method), or IR. In addition, the deacetylation of a composition in solution may be measured via methods known in the art, such as, CZE (capillary zone electrophoresis), GC-MS, ion chromatography, and SEC-UV.

Dose: As used herein, the term “dose” means a quantity of an agent, ABI, API, formulation, or pharmaceutical composition administered or recommended to be administered at a particular time.

HPV and PV: As used herein, the terms “HPV” and “PV” refer to human papillomavirus and papillomavirus, respectively.

MW: As used herein, the term “MW” refers to the average molecular weight.

Patient (used interchangeably with “subject” herein): As used herein, the term “patient” refers to any human being that is to receive the vaccines or pharmaceutical compositions described herein. As defined herein, “patient” includes those already infected with one or more pathogens or infectious agent (e.g. one or more types of HPV) as well as those in which infection with a pathogen or infectious agent (e.g. one or more types of HPV) is to be prevented. The term “patient” or “subject” may also refer to an organism, typically a mammal (e.g., a human, in some embodiments including prenatal human forms). In some embodiments, a patient is suffering from a relevant disease, disorder or condition. In some embodiments, a subject is susceptible to a disease, disorder, or condition. In some embodiments, a patient displays one or more symptoms or characteristics of a disease, disorder or condition. In some embodiments, a patient does not display any symptom or characteristic of a disease, disorder, or condition. In some embodiments, a patient is someone with one or more features characteristic of susceptibility to or risk of a disease, disorder, or condition. In some embodiments, a patient is an individual to whom diagnosis and/or therapy is and/or has been administered. As used herein, the term “patient in need thereof” means a human or animal patient in which a disease, disorder, or clinical manifestation thereof is to be prevented, such patient to be the subject of a prophylactic treatment described herein.

Pharmaceutically acceptable: As used herein with respect to a carrier, diluent, or excipient of a pharmaceutical composition, the term “pharmaceutically acceptable” indicates that a carrier, diluent, or excipient must be compatible with the other ingredients of the composition and not deleterious to the recipient thereof.

Pharmaceutical composition: As used herein, the term “pharmaceutical composition,” refers to a composition containing an active pharmaceutical or biological ingredient, along with one or more additional components, e.g., a composition in which an active agent is formulated together with one or more pharmaceutically acceptable carriers. As used herein, the terms “pharmaceutical formulation” and “formulation” are used interchangeably with “pharmaceutical composition.” In some embodiments, the active agent is present in a unit dose amount appropriate for administration in a therapeutic regimen that shows a statistically significant probability of achieving a predetermined therapeutic effect when administered to a relevant population. The pharmaceutical compositions or formulations can be liquid or solid (e.g., lyophilized). Additional components that may be included as appropriate include pharmaceutically acceptable excipients, additives, diluents, buffers, sugars, amino acids, chelating agents, surfactants, polyols, bulking agents, stabilizers, lyo-protectants, solubilizers, emulsifiers, salts, adjuvants, tonicity enhancing agents, delivery vehicles, and anti-microbial preservatives. The pharmaceutical compositions or formulations are nontoxic to recipients at the dosages and concentrations employed. In some embodiments, a pharmaceutical composition is specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, the term formulation refers to a dose of a vaccine, which can be included in any volume suitable for injection.

Quaternization: As used herein, the term “quaternization” refers to an atom that has chemical bonds to four other atoms and is considered to have a positive electrostatic charge. In some embodiments, quaternization refers to the ratio of the number of nitrogen atoms of the trimethyl chitosan (TMC) bearing three methyl groups to the total number of nitrogen atoms of the TMC. The degree of quaternization may be expressed as a ratio or percentage, e.g., TMC has a degree of quaternization of about 30% to about 70%.

RSV: As used herein, the term “RSV” refers to Respiratory Syncytial Virus.

Therapeutically Effective Amount: As used herein, the term “therapeutically effective amount” refers to an amount of the ABI or API (e.g. protein, peptide, or VLP) sufficient to produce the desired therapeutic effect in a human or animal, e.g., the amount necessary to elicit an immune response, treat, cure, prevent, or inhibit development and progression of a disease or the symptoms thereof and/or the amount necessary to ameliorate symptoms or cause regression of a disease. Therapeutically effective amount may vary depending on the structure and potency of the active ingredient and the contemplated mode of administration. One of skill in the art can readily determine a therapeutically effective amount of a given ABI or API.

Trimethyl chitosan: As used herein, the term “trimethyl chitosan” or “TMC” refers to an N-quaternary chitosan derivative. Examples of trimethyl chitosan include a high molecular weight TMC having a degree of quaternization of about 30-70%, e.g., Sigma-Aldrich Product Number 912034, a medium molecular weight TMC having a degree of quaternization of about 40-60%, e.g., Sigma-Aldrich Product Number 912123, and a low molecular weight TMC having a degree of quaternization of 50% or greater, e.g., Sigma-Aldrich Product Number 912700.

Vaccine: As used herein, the term “vaccine” or “vaccine composition” refers to a substance or preparation used to stimulate the production of antibodies and provide immunity against one or more diseases pathogens, or infectious agents, prepared from the causative agent of a disease, its products, or a synthetic substitute, treated to act as an antigen without inducing the disease. A vaccine composition may include at least one ABI (e.g., antigen or VLP) in a pharmaceutically acceptable vehicle useful for inducing an immune response in a subject. A vaccine composition is administered by doses and techniques known to those skilled in the pharmaceutical or veterinary fields, considering factors such as the age, sex, weight, species, and condition of the recipient animal and the route of administration.

Valent: As used herein, the term “valent” refers to the presence of a specified number of antigens in a vaccine. For example, the terms bi-valent, bivalent, 2 valent, or 2-valent refer to two different antigens. Similarly, the terms quadrivalent, 4 valent, or 4-valent refer to four different antigens and the terms nonavalent, 9 valent or 9-valent refer to nine different antigens.

Viscosity: As used herein, viscosity refers to the measure of a substance's resistance to deformation or flow at a given rate. Viscosity can be measured, for example, by using a viscometer at a given shear rate or shear rates that are appropriately selected by those skilled in the art to accurately measure viscosity in the viscosity range of the sample of interest. The viscosity may be measured by any method or instrument for measuring viscosity known in the art. For example, in some embodiments, the viscosity of the substances may be measured using a viscometer (e.g., Brookfield DVII+pro) at 20° C. at a standard concentration (e.g., 1% in 1% acetic acid). In other embodiments, the viscosity may be measured using a Rheosense microVisc capillary viscometer. In some embodiments, the viscosity is a measure of a solid, such as e.g., chitosan, dissolved in a 1% acetic acid solution to achieve a final chitosan concentration of 1% (w/v).

Virus Like Particles: As used herein, the term “virus like particles” or “VLPs” refers to agents that are morphologically similar to authentic virions or provide an arrayed display of an antigen and are capable of inducing high antibody neutralization titers after administration in an animal. VLPs lack the viral genetic material of the authentic virions and are thus non-infectious.

Water-Soluble Chitosan: As used herein, the term “water-soluble chitosan” refers to a chitosan that is prepared by solubilizing a chitosan powder in water or aqueous buffer. Examples of water-soluble chitosan include chitosan hydrochloride, chitosan chloride, chitosan ascorbate, carboxylic acid salts of chitosan, and the like, e.g., Heppe Medical Chitosan Item Numbers 54046 and 54047.

### BRIEF DESCRIPTION OF THE DRAWINGS

FIG. 1 shows a graphical depiction of the viscosity of chitosan of different average molecular weights and concentrations. A discussion of FIG. 1 is found in Example 4.

FIGS. 2A and 2B show a graphical depiction of the thermal stability of the average molecular weight of chitosan samples in buffer, as further explained in Example 4. FIG. 2A depicts stability of acid soluble chitosan of approximately 20 kDa. FIG. 2B depicts stability of water-soluble chitosan of approximately 60 kDa.

FIGS. 3A and 3B show a graphical depiction of the average molecular weight of chitosan samples combined with a 9 valent HPV vaccine, as further explained in Example 4. FIG. 3A depicts stability of acid soluble chitosan of approximately 20 kDa. FIG. 3B depicts stability of water-soluble chitosan of approximately 60 kDa.

FIG. 4 shows a graphical depiction of High Performance Size Exclusion Chromatography (HPSEC) chromatograms of samples from chitosan cleavage, as described in Example 5.

FIG. 5 shows a graphical depiction of the reduction in chitosan molecular weight with chitosanase treatment time, as described in Example 5.

FIG. 6 shows a graphical depiction of modeling of the interaction of average molecular weight and concentration on chitosan viscosity, as described in Example 5.

FIG. 7 shows a graphical depiction of modeling of predicted viscosity as a function of molecular weight, as described in Example 5.

FIG. 8 shows a graphical depiction of chitosan adjuvant effect in mice vaccinated with an RSV antigen, as described in Example 6.

FIG. 9 shows a graphical depiction of chitosan adjuvant effect in mice vaccinated with an RSV antigen, as described in Example 6.

FIG. 10 shows a graphical depiction of modeling of the viscosity of trimethyl chitosan solutions (shown using numeric text as symbols) overlaid on the data and model for the viscosity of chitosan (from FIG. 6) and is described in Example 7.

FIGS. 11A-11D show a graphical depiction of temperature stability studies of viscosity for chitosan and trimethyl chitosan samples at 2-8 C (FIG. 11A), 25 C (FIG. 11B), 37 C (FIG. 11C), and 45 C (FIG. 11D) and is described in Example 8.

FIGS. 12A-12D show a graphical depiction of temperature stability studies of molecular weight for chitosan and trimethyl chitosan samples at 2-8° C. (FIG. 12A). 25° C. (FIG. 12B), 37° C. (FIG. 12C), and 45° C. (FIG. 12D). Each figure is described further in Example 8.

FIG. 13 shows a graphical depiction of the chitosan and trimethyl chitosan adjuvant effect in mice vaccinated with an RSV antigen, as described in Example 9.

FIG. 14 shows a graphical depiction of serum neutralizing antibody titers at week 5 in mouse vaccination study with an RSV antigen.

### DETAILED DESCRIPTION

In one aspect, the invention comprises a vaccine adjuvant that includes chitosan, or trimethyl chitosan, or other chitosan derivatives (in soluble form) that has relatively lower viscosity and/or relatively higher solubility when compared to other forms of chitosan. As is known to one of skill in the art, chitosan or chitosan derivatives having high viscosity and/or low solubility can pose challenges for vaccine manufacturing processes, analytical testing, formulation, storage, and administration (i.e., syringeability, injectability, etc.) to a recipient. By selecting a chitosan or a chitosan derivative that is relatively lower in viscosity and/or higher in solubility, these challenges may be overcome. Importantly, these soluble and/or lower viscosity chitosan or chitosan derivatives should maintain adjuvant activity, preferentially comparable adjuvant activity to higher viscosity chitosan at an achievable dose level.

The invention is based, in part, on the discovery that reducing the average molecular weight of chitosan led to the benefit of lower viscosity while retaining adjuvant activity, including reducing to a size below about 15 kDa average molecular weight (measured by High Performance Size Exclusion Chromatography-Multi Angle Light Scattering—Refraction Index (“HPSEC-MALS-RI”)). Using trimethyl chitosan samples with different molecular weights spanning a wide range of molecular weights, it was shown herein that the chitosan derivative trimethyl chitosan is more soluble, particularly near and above physiological pH, than chitosan, is less viscous at equivalent concentration than chitosan, and has comparable immunogenicity in a mouse immunogenicity study. See. e.g., Examples 6 and 9.

### Chitosan and Chitosan Derivatives

Chitin, a polymer of N-acetylglucosamine (i.e., (1→4)-2-acetamido-2-deoxyβ-d-glucan), is a significant component of the body of all crustaceans and is also present in the exoskeleton and the cell wall of fungi, insects, and yeast. Chitosan. i.e., α-(1→4)-2-amino-2-deoxy-β-d-glucan, is the mostly deacetylated form of the naturally occurring polysaccharide chitin. Chitosan is typically formed by deacetylation of chitin in the presence of alkali. The term “chitosan” refers to the class of molecules having a degree of deacetylation that is different from chitin. For example, molecules having a deacetylation below 75% are consider chitin. In contrast, molecules having a deacetylation above 75% are considered chitosan. The deacetylation of chitosan powder may be measured via NMR, UV (EP method), or IR. The deacetylation of chitosan in solution may be measured via CZE (capillary zone electrophoresis), GC-MS (gas chromatography-mass spectrometry), ion chromatography, and SEC-UV (size-exclusion chromatography by UV/Vis detection).

In some embodiments, the chitosan adjuvant includes a chitosan. In some embodiments, the chitosan adjuvant includes a chitosan having a deacetylation of 75% or greater. In some embodiments, the chitosan adjuvant includes chitosan that has a degree of deacetylation in the range of about 75-99%. In some embodiments, the chitosan adjuvant includes chitosan that has a degree of deacetylation in the range of about 85-99%. In some embodiments, the chitosan adjuvant includes chitosan that has a degree of deacetylation greater than 90%. In some embodiments, the chitosan adjuvant includes chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. Deacetylation may be calculated according to any of the methods described herein.

In some embodiments, the chitosan adjuvant includes a water-soluble chitosan having a degree of deacetylation in the range of about 75-99%. In some embodiments, the chitosan includes water-soluble chitosan that has a degree of deacetylation in the range of about 75-99%. In some embodiments, the chitosan includes water-soluble chitosan that has a degree of deacetylation in the range of about 85-99%. In some embodiments, the chitosan includes water-soluble chitosan that has a degree of deacetylation greater than 90%. In some embodiments, the chitosan includes a water-soluble chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. In some embodiments, the water-soluble chitosan is chitosan hydrochloride. Deacetylation may be calculated according to any of the methods described herein.

In some embodiments, the chitosan adjuvant includes an acid-soluble chitosan that has a degree of deacetylation in the range of about 75-99%. In some embodiments, the acid-soluble chitosan is chitosan hydrochloride. In some embodiments, the chitosan includes an acid-soluble chitosan that has a degree of deacetylation in the range of about 85-99%. In some embodiments, the chitosan includes an acid-soluble chitosan that has a degree of deacetylation greater than or equal to 90%. In some embodiments, the chitosan includes an acid-soluble chitosan that has a degree of deacetylation of about 75% or more. 80% or more, 85% or more. 90% or more, 91% or more, 92% or more, 93% or more, 94% or more, 95% or more, 96% or more, 97% or more, 98% or more, or 99% or more. In some embodiments, the chitosan includes an acid-soluble chitosan that has a degree of deacetylation of about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%. Deacetylation may be calculated according to any of the methods described above.

In some embodiments, the chitosan adjuvant includes a chitosan derivative. In some embodiments, the chitosan derivative is an N-quaternary chitosan derivative. In some embodiments, the chitosan derivative is trimethyl chitosan or TMC. In some embodiments, the TMC has an average molecular weight of between about 225 kDa and about 275. In some embodiments, the TMC has an average molecular weight of between about 125 kDa and about 160 kDa. In some embodiments, the TMC has an average molecular weight of between about 100 kDa and about 150 kDa.

In some embodiments, the chitosan derivative includes a trimethyl chitosan (TMC) that has a degree of quaternization in the range of about 25-99%. In some embodiments, the chitosan includes TMC that has a degree of quaternization in the range of about 25-75%. In some embodiments, the chitosan derivative includes TMC that has a degree of quaternization in the range of about 35-65%. In some embodiments, the chitosan derivative includes TMC that has a degree of quaternization of 50% or greater. In some embodiments, the chitosan derivative includes TMC that has a degree of quaternization of about 30%, 31%, 32%, 33%, 34%, 35%, 36%, 37%, 38%, 39%, 40%, 415, 42%, 43%, 44%, 45%, 46%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the TMC has an average molecular weight of between about 225 kDa and about 275 kDa and a degree of about quaternization of 30-70%. In some embodiments, the TMC has an average molecular weight of about 125 kDa and about 160 kDa and a degree of about quaternization of 40-60%. In some embodiments, the TMC has an average molecular weight of between about 100 kDa and about 150 kDa and a degree of about quaternization of equal to or greater than 50%.

In some embodiments, the chitosan or chitosan derivative has a viscosity in the range of about 1 cP to about 200 cP when measured with a viscometer (e.g. Brookfield DVII+pro) at 20° C. at a standard concentration (e.g. 1% in 1% acetic acid or the viscosity is a measure of chitosan dissolved in a 1% acetic acid solution to achieve a final chitosan concentration of 1% weight per volume (w/v)). In some embodiments, the viscosity of chitosan or chitosan derivatives is measured by any of several methods known in the art, including but not limited to the use of a Brookfield viscometer or a Rheosense viscometer, and at concentrations and in formulations other than 1% chitosan in 1% acetic acid. In some embodiments, the chitosan or chitosan derivative has a viscosity in the range of about 1 cP to about 100 cP. In some embodiments, the chitosan or chitosan derivative has a viscosity in the range of about 5 cP to about 100 cP. In some embodiments, the chitosan or chitosan derivative has viscosity in the range of about 1 cP to about 50 cP. In some embodiments, the chitosan or chitosan derivative has a viscosity in the range of about 5 cP to about 50 cP. In some embodiments, the chitosan or chitosan derivative has a viscosity in the range of about 1 cP to about 25 cP. In some embodiments, the chitosan or chitosan derivative has a viscosity in the range of about 5 cP to about 25 cP. In some embodiments, the chitosan has a viscosity in the range of about 1 cP to about 20 cP. In some embodiments, the chitosan or chitosan derivative has viscosity in the range of about 5 cP to about 20 cP. In some embodiments, the chitosan or chitosan derivative has a viscosity in the range of about 1 cP to about 15 cP. In some embodiments, the chitosan or chitosan derivative has a viscosity in the range of about 5 cP to about 15 cP. In some embodiments, the chitosan or chitosan derivative has a viscosity in the range of about 1 cP to about 10 cP. In some embodiments, the chitosan or chitosan derivative has a viscosity in the range of about 5 cP to about 10 cP. In some embodiments, the chitosan or chitosan derivative has a viscosity of equal to or less than 100 cP. In some embodiments, the chitosan or chitosan derivative has a viscosity of equal to or less than 75 cP. In some embodiments, the chitosan or chitosan derivative has a viscosity of equal to or less than 50 cP. In some embodiments, the chitosan or chitosan derivative has a viscosity of equal to or less than 40 cP. In some embodiments, the chitosan or chitosan derivative has a viscosity of equal to or less than 30 cP. In some embodiments, the chitosan or chitosan derivative has a viscosity of equal to or less than 20 cP. In some embodiments, the chitosan or chitosan derivative has a viscosity of equal to or less than 15 cP. In some embodiments, the chitosan or chitosan derivative has a viscosity of equal to or less than 10 cP. In some embodiments, the chitosan or chitosan derivative has a viscosity of equal to or less than 5 cP. In some embodiments, the chitosan or chitosan derivative has viscosity of 1 cP. 2 cP, 3 cP, 4 cP, 5 cp, 6 cp, 7 cP. 8 cP, 9 cP, 10 cP, 11 cP, 12 cP, 13 cP. 14 cP, 15 cP, 16 cP, 17 cP, 18 cP, 19 cP, 20 cP. 21 cP, 22 cP. 23 cP, 24 cP. 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, or 50 cP.

In some embodiments, the chitosan or chitosan derivative has a molecular weight of about 10 kDa to about 300 kDa. In some embodiments, the chitosan or chitosan derivative has a molecular weight of about 10 kDa to about 50 kDa. In some embodiments, the chitosan or chitosan derivative has a molecular weight of about 50 kDa to about 100 kDa. In some embodiments, the chitosan or chitosan derivative has a molecular weight of about 100 kDa to about 275 kDa. In some embodiments, the chitosan or chitosan derivative has a molecular weight of about 50 kDa to about 150 kDa. In some embodiments, the chitosan or chitosan derivative has a molecular weight of about 100 kDa to about 200 kDa. In some embodiments, the chitosan or chitosan derivative has a molecular weight of about 20 kDa. In some embodiments, the chitosan or chitosan derivative has a molecular weight of about 80 kDa. In some embodiments, the chitosan or chitosan derivative has a molecular weight of about 60 kDa-120 kDa. In some embodiments, the chitosan or chitosan derivative has a molecular weight of about 100-120 kDa. In some embodiments, the chitosan or chitosan derivative has a molecular weight of about 130-150 kDa. In some embodiments, the chitosan or chitosan derivative has a molecular weight of about 250-270 kDa.

In some embodiments, the chitosan or chitosan derivative is present in an amount of about 0.1 μg to about 200 mg. In some embodiments, the chitosan or chitosan derivative is present in an amount of about 0.1 μg to about 100 mg. In some embodiments, the chitosan or chitosan derivative is present in an amount of about 0.1 μg to about 50 mg. In some embodiments, the chitosan or chitosan derivative is present in an amount of about 0.1 μg to about 25 mg. In some embodiments, the chitosan or chitosan derivative is present in an amount of about 0.1 μg to about 20 mg. In some embodiments, the chitosan or chitosan derivative is present in an amount of about 0.1 μg to about 100 mg. In some embodiments, the chitosan or chitosan derivative is present in an amount of less than about 100 mg. In some embodiments, the chitosan or chitosan derivative is present in an amount of less than about 90 mg. In some embodiments, the chitosan or chitosan derivative is present in an amount of less than about 80 mg. In some embodiments, the chitosan or chitosan derivative is present in an amount of less than about 70 mg. In some embodiments, the chitosan or chitosan derivative is present in an amount of less than about 60 mg. In some embodiments, the chitosan or chitosan derivative is present in an amount of less than about 50 mg. In some embodiments, the chitosan or chitosan derivative is present in an amount of less than about 40 mg. In some embodiments, the chitosan or chitosan derivative is present in an amount of less than about 30 mg. In some embodiments, the chitosan or chitosan derivative is present in an amount of less than about 20 mg. In some embodiments, the chitosan or chitosan derivative is present in an amount of less than about 10 mg. In some embodiments, the chitosan or chitosan derivative is present in an amount of less than about 5 mg.

In some embodiments, the chitosan or chitosan derivative is combined with a buffer. In some embodiments, the buffer is selected from any pharmaceutically acceptable buffer, including acetic acid, histidine, citrate. Bis-Tris. HEPES, phosphate, MES, and combinations thereof. In some embodiments, the buffer is present in an amount of 1 nmMol to about 100 mMol.

In some embodiments, the chitosan or chitosan derivative is combined with a tonicity modifier. In some embodiments, the tonicity modifier is selected from any pharmaceutically acceptable tonicity modifiers, such as sodium chloride, potassium chloride, sucrose, trehalose and combinations thereof. In some embodiments the tonicity modifier is present in an amount of 10 mM to 500 mM.

In some embodiments, the chitosan or chitosan derivative is combined with a detergent. In some embodiments, the detergent is selected from any pharmaceutically acceptable detergent, such as Polysorbate 80, Polysorbate 20, Poloxamer 188, and combinations thereof. In some embodiments, the detergent is present in an amount of 0.001 to 0.2% (w/v).

In some embodiments, the water-soluble chitosan is formed, for example, by placing approximately 5-5000 mL water in a 10-10000 mL volumetric flask and adding approximately 0.01 to 500 g grams of a buffer, such as histidine, and stirring the combination until the solids are dissolved. After the histidine is in solution, approximately 0.001 to 1000 grams of a water-soluble chitosan, such as chitosan hydrochloride is added to the histidine solution. The combination is then mixed until the water-soluble chitosan solids are dissolved. After the water-soluble chitosan is completely in solution, the flask is then filled to the volumetric line with Q.S. HPLC water to a target volume of 10 to 10000 mL. The pH of the resulting solution is then tested to verify pH. In some embodiments, the pH range of the resulting solution is 5.0-6.5. In some embodiments, the pH range of the resulting solution is 5.3 to 6.2. In some embodiments, the pH range of the resulting solution is 5.5 to 6.0. In some embodiments, the pH range of the resulting solution is 5.6 to 5.9. The water-soluble chitosan solution is then sterile filtered in a sterile biosafety cabinet using syringes and a syringe filter system, such as, a Sterile Acrodisc) Syringe Filter with a Supor® Membrane (Pall® Corporation) to sterile filter the solution. Alternatively, an appropriately sized capsule filter with PES membrane can be used for sterile filtration in a biological safety cabinet.

In some embodiments, the acid-soluble chitosan is formed, for example, by placing approximately 5 to 5,000 mL of a buffer, such as 1% acetic acid, in a 10 to 10,000 mL volumetric flask and adding approximately 0.001 to 1,000 grams of an acid-soluble chitosan and stirring the combination until the chitosan solids are dissolved. After the acid-soluble chitosan is in solution, solid powder of a buffer, such as histidine, is weighed to achieve a final solution concentration of 10 to 500 mM, which may be added to the acid-soluble chitosan solution. In some embodiments, the pH of the acid-soluble chitosan solution is adjusted to a range of approximately 5.0-6.5 using either an amount of buffer, such as histidine, or using a basic solution such as a 20% w/v sodium hydroxide solution. In some embodiments, the pH range of the resulting solution is 5.3 to 6.2. In some embodiments, the pH range of the resulting solution is 5.5 to 6.0. In some embodiments, the pH range of the resulting solution is 5.6 to 5.9. The acid-soluble chitosan solution is then sterile filtered in a sterile biosafety cabinet using syringes and a syringe filter system, such as, a Sterile Acrodisc® Syringe Filter with a Supor® Membrane (Pall® Corporation) to sterile filter the solution. Alternatively, an appropriately sized capsule filter with PES membranes can be used for sterile filtration in a biological safety cabinet.

In some embodiments, the trimethyl chitosan is prepared by dissolving samples of trimethyl chitosan having “low molecular weight” (e.g., LMW, Millipore Sigma product 912700), “medium molecular weight” (e.g., MMW, Millipore Sigma product 912123) and “high molecular weight” (e.g., HMW, Millipore Sigma product 912034) trimethyl chitosan in water. In some embodiments, the samples were sterile filtered (0.22 micron).

In some embodiments, the chitosan derivative has a viscosity of at least 10 times less than the chitosan, wherein the chitosan derivative and chitosan have approximately the same concentration and molecular weight. In some embodiments, the chitosan derivative has a viscosity of at least 10 times less than the chitosan, wherein the chitosan derivative and chitosan have approximately the same molecular weight. In some embodiments, the chitosan derivative has a viscosity of at least 10 times less than the chitosan, wherein the chitosan derivative has a higher molecular weight than the chitosan.

### RSV Antigens

Respiratory Syncytial Virus (RSV) is a member of the pneumo virus family. RSV infection is the leading cause of lower respiratory tract infection in both young children and older adults (>65 years). The envelope of RSV contains three surface glycoproteins: F, G, and SH. The G and F proteins are protective antigens and targets of neutralizing antibodies. The F protein, however, is more conserved across RSV strains and types (A and B). RSV F is a type 1 viral fusion protein which structurally rearranges from a metastable prefusion form to a highly stable postfusion form. Although targets for neutralizing monoclonal antibodies exist on the postfusion conformation of F protein, the neutralizing Ab response primarily targets the F protein prefusion conformation in people naturally infected with RSV (Magro M et al., Proc Natl Acad Sci USA 109(8):3089-94, 2012; Ngwuta J O et al., Sci Transl Med 7(309):309ral62, 2015). Therefore, engineered RSV F protein stabilized in its prefusion conformation has been an attractive strategy for developing RSV F vaccine antigens. For example, a recombinant RSV F trimer including the “DS-Cav1” substitutions (155C, 290C, 190F, and 207L) was previously shown to elicit neutralizing immune response in animal models that is greater than the response observed for post-fusion F based RSV immunogens (McLellan et al., Science, 342: 592-598, 2013).

RSV F protein is a type I fusion glycoprotein that is well conserved between clinical isolates, including between the RSV-A and RSV-B antigenic subgroups. The F protein transitions between prefusion and more stable postfusion states, thereby facilitating entry into target cells. RSV F glycoprotein is initially synthesized as an F0 precursor protein. RSV F0 folds into a trimer, which is activated by furin cleavage into the mature prefusion protein comprising F1 and F2 subunits (Bolt, et al, Virus Res., 68:25, 2000). RSV F protein stabilized in the prefusion conformation produces a greater neutralizing immune response in animal models than that observed with RSV F protein stabilized in the post fusion conformation (McLellan et al, Science, 342: 592-598, 2013). As such, stabilized prefusion RSV F proteins are good candidates for inclusion in an RSV vaccine. Soluble RSV ectodomains stabilized in the prefusion conformation have previously been generated, including the “DS-Cav1” substitutions. See, WO 2014/160463A1. WO 2017/172890A1, and WO 2019/147749A2, the contents of each of which are hereby incorporated by reference.

In some embodiments, the ABI includes an RSV antigen, such as an RSV subunit pre-F protein vaccine, DS-Cav1 substitutions, similar to what has been previously described by McLellan J S. et al., Structure-based design of a fusion glycoprotein vaccine for respiratory syncytial virus. Science 2013 Nov. 1; 342(6158):592-8. The DS-Cav1 and variant RSV F sequences were codon optimized for mammalian codon usage (Life Technologies), cloned into an expression vector, and transiently transfected into Expi293 suspension cells (Life Technologies).

In some embodiments, the ABI includes an RSV antigen such as that described in U.S. Patent Publication No. 2021/0300971, filed Jan. 24, 2019, the contents of which are incorporated by reference.

### The RSV Vaccine Compositions

In some embodiments, a pharmaceutical composition (i.e., a composition that includes an ABI or API and a pharmaceutically acceptable carrier) is provided that includes a chitosan or chitosan derivative and an ABI that includes an RSV antigen.

The vaccines of the invention comprise the antigenic determinants required to induce the generation of neutralizing antibodies in the subject. The vaccines are expected to be sufficiently safe to be administered without the risk of clinical infection, have no toxic side effects, are stable, compatible with conventional carriers and can be administered effectively. In some embodiments, a chitosan or chitosan derivative of the invention may be combined with an RSV vaccine.

Pharmaceutical compositions, formulations, and vaccines of the invention may be administered subcutaneously, topically, orally, on the mucosa, intravenously, or intramuscularly. The pharmaceutical compositions, formulations, and vaccines are administered in an amount sufficient to elicit a protective immune response. Vaccines, pharmaceutical compositions and formulations can be administered by various routes, for example, orally, parenterally, subcutaneously, on the mucosa, or intramuscularly. The dose administered may vary depending on the general condition, sex, weight and age of the patient, the route of administration and the type of antigen in the vaccine. The vaccine, pharmaceutical composition, or formulation may be in the form of a capsule, suspension, elixir or solution. It may be formulated with an immunologically acceptable carrier.

### HPV VLPs

Human papillomaviruses (HPVs) are small, double-stranded DNA viruses that infect the skin and internal squamous mucosal epithelia of men and women. HPVs are classified based on their carcinogenic properties. HPVs include major (L1) and minor (L2) capsid proteins. Over 200 distinct HPV genotypes have been identified (Li et al., “Rational design of a triple-type human papillomavirus vaccine by compromising viral-type specificity,” Nature, 9:5360 (2018)), many of which have been associated with pathologies ranging from benign proliferative warts to malignant carcinomas of the cervix (for review, see McMurray et al., Int. J. Exp. Pathol. 82(1): 15-33 (2001)). Those HPV types considered to be “high-risk” include HPV types 16, 18, 31, 33, 35, 39, 45, 51, 52, 56, 58, 59, 68 and 73. (Chan et al., “Human Papillomavirus Infection and Cervical Cancer: Epidemiology, Screening, and Vaccination-Review of Current Perspectives,” Journal of Oncology, vol. 2019, Article ID 3257939, 2019.)

HPV is the primary etiological agent of cervical cancer, one of the most common cancer types in women, as well as squamous cell carcinomas of the anus, tonsil, tongue, vulva, vagina, and penis. HPV16 and HPV18 are well known as the most virulent of the high-risk HPV types as they cause approximately 70% of all invasive cervical cancer in the world.

Papillomaviruses are small (50-60 run), nonenveloped, icosahedral DNA viruses that encode up to eight early (E1-E7) and two late (L1-L2) genes. The L1 protein is the major capsid protein and has a molecular weight of 55-60 kDa. Expression of the L1 protein or a combination of the L1 and L2 proteins in yeast, insect cells, mammalian cells or bacteria leads to self-assembly of virus-like particles (VLPs) (for review, see Schiller and Roden, in Papillomavirus Reviews: Current Research on Papillomaviruses; Lacey, ed. Leeds, UK: Leeds Medical Information, pp 101-12 (1996)).

VLPs are morphologically similar to authentic virions and are capable of inducing high titers of neutralizing antibodies upon administration into animals or humans. Because VLPs do not contain the potentially oncogenic viral genome, they present a safe alternative to the use of live virus in HPV vaccine development (for review, see Schiller and Hidesheim, J Clin. Virol. 19: 67-74 (2000)). For this reason, the L1 and L2 genes have been identified as immunological targets for the development of prophylactic and therapeutic vaccines for HPV infection and disease.

VLP-based vaccines have proven to be effective at inducing immune responses in human subjects vaccinated with bivalent HPV 16 and 18 (Harper et al. Lancet 364 (9447): 1757-65 (2004)), quadrivalent HPV 6, 11, 16, and 18 (Villa et al. Vaccine 24: 5571-5583 (2006)) and multi-valent HPV 6, 11, 16, 18, 31, 33, 45, 52 and 58 VLP-based vaccines. Three approved VLP-based vaccines against HPV are administered according to 2 or 3 dose regimens. CERVARIX® (GlaxoSmithKline Biologicals. Rixensart, Belgium), is a bivalent vaccine protective against HPV 16 and 18. GARDASIL® and GARDASIL® 9 (Merck & Co., Inc., Rahway, NJ, USA) protect against two and seven additional HPV types, respectively, and prevent additional HPV-related anogenital diseases, including wart formation. The additional five high-risk strains in GARDASIL® 9 compared to GARDASIL® increase protection from about 70% of anogenital malignancies to about 90%. (Id., M. Nygard, et al., “Evaluation of the long-term anti-human papillomavirus (HPV) 6, 11, 16, and 18 immune responses generated by the quadrivalent HPV vaccine,” Clinical and Vaccine Immunology, vol. 22, no. 8, pp. 943-948, 2015.)

As stated above, the pharmaceutical compositions and formulations of the invention may include at least one HPV VLP type, such as HPV 16 or 18. In particular embodiments of the compositions disclosed herein, the vaccine further comprises VLPs of at least one additional HPV type. In further embodiments, the at least one additional HPV type is selected from the group consisting of: 6, 11, 16, 18, 26, 31, 33, 35, 39, 45, 51, 52, 53, 55, 56, 58, 59, 66, 68, 69, 70, 73, and 82. In some embodiments, the at least one additional HPV type includes HPV 16 and 18. In some embodiments, the at least one additional HPV type includes HPV 6, 11, 16, and 18. In some embodiments, the at least one additional HPV type includes HPV 6, 18, 52, and 58. In some embodiments, the at least one additional HPV type includes HPV 6, 11, 16, 18, 31, 45, 52, and 58. In some embodiments, the at least one additional HPV type includes HPV 6, 11, 16, 18, 33, 45, 52, and 58. In some embodiments, the at least one additional HPV type includes HPV 6, 11, 16, 18, 31, 33, 45, 52, and 58. In some embodiments, the at least one additional HPV type includes 6, 11, 16, 18, 31, 33, 45, 52, and 59. In some embodiments, the at least one additional HPV type includes HPV 6, 11, 16, 18, 31, 33, 45, 53, and 58. In some embodiments, the at least one additional HPV type includes HPV 6, 11, 16, 18, 31, 33, 45, 53, and 59. In some embodiments, the at least one additional HPV type includes HPV 6, 11, 16, 18, 31, 33, 35, 45, 52, and 58. In some embodiments, the at least one additional HPV type includes HPV 6, 11, 16, 18, 31, 33, 35, 45, 52, 58, and 59. In some embodiments, the at least one additional HPV type includes HPV 6, 11, 16, 18, 31, 33, 45, 52, 58, 59, and 68. In some embodiments, the at least one additional HPV type includes HPV 6, 11, 16, 18, 31, 33, 35, 39, 45, 51, 52, 56, 58, and 59. In some embodiments, the at least one additional HPV type includes HPV 6, 11, 16, 18, 26, 31, 33, 35, 45, 51, 52, 58, 59, and 69. In some embodiments, the at least one additional HPV type includes HPV 6, 11, 16, 18, 26, 31, 33, 35, 39, 45, 51, 52, 58, 59, 68, 69, and 70. In some embodiments, the at least one additional HPV type includes HPV 6, 11, 16, 18, 26, 31, 33, 35, 39, 45, 51, 52, 53, 56, 58, 59, 66, 68, 69, and 70. In some embodiments, the invention provides pharmaceutical compositions comprising HPV VLPs comprised of recombinant L1 or recombinant L1+L2 proteins of HPV. HPV L1 or L1+L2 protein can be expressed recombinantly by molecular cloning of L1 or L1+L2 DNA into an expression vector containing a suitable promoter and other appropriate transcription regulatory elements, and transferring into prokaryotic or eukaryotic host cells to produce recombinant protein. Techniques for such manipulations are fully described by Sambrook et al. (Molecular Cloning: A Laboratory Manual: Cold Spring Harbor Laboratory, Cold Spring Harbor, New York, (1989)), which is hereby incorporated by reference. VLPs can self-assemble when L1 protein is recombinantly expressed in a host cell.

The recombinant HPV L1 proteins of the compositions of the invention may be any full-length L1 protein sequence that can be found in nature or any mutated or truncated L1 protein that is capable of self-assembling into VLPs. In particular embodiments, the pharmaceutical compositions and vaccines described herein comprise HPV VLPs comprised of recombinant HPV L1 protein and do not contain HPV L2 protein. In certain embodiments, the vaccine compositions or pharmaceutical compositions described herein comprise HPV VLPs comprised of a full-length recombinant HPV L1 protein. In other embodiments, the HPV VLPs are comprised of truncated HPV L1 protein, e.g., L1 protein that are truncated at the C-terminal end. L1 protein sequences for use in the invention can be determined by isolating DNA from one or more clinical samples containing an HPV type of choice, determining the sequence of the HPV L1 DNA sequence, and translating the DNA sequence into an amino acid sequence using the genetic code. Many exemplary L1 sequences suitable for use in the invention can be found in the literature. See, e.g., U.S. Pat. Nos. 5,820,870; 7,250,170; 7,276,243; 7,482,428; 7,976,848; 7,498,036; 7,700,103; 7,744,892; and 5,437,951; Kirii et al. (Virology 185(1): 424-427 (1991)). Further L1 proteins that are useful in the compositions and formulations of the invention include biologically active fragments and/or mutants of an HPV L1 sequence, including but not necessarily limited to amino acid substitutions, deletions, additions, amino terminal truncations and carboxy-terminal truncations, such that these mutations provide for L1 proteins or protein fragments that are capable of forming a VLP. See. e.g., WO 2006/114312 and U.S. Pat. No. 6,599,508. Appropriate host cells for the expression of recombinant HPV L1 or recombinant L1+L2 and subsequent self-assembly of VLPs include, but are not limited to yeast cells, insect cells, mammalian cells or bacteria. In exemplary embodiments of the invention, the VLPs are produced in yeast cells such as a yeast selected from the group consisting of: Saccharomyces cerevisiae, Hansenula polymorpha, Pichia pastoris, Kluyveromyces fragilis, Kluyveromyces lactis, and Schizosaccharomyces pombe. In particular embodiments, the HPV VLPs are produced in Saccharomyces cerevisiae cells. Expression of HPV VLPs in yeast cells offers the advantages of being cost-effective and easily adapted to large-scale growth in fermenters.

The invention also includes pharmaceutical compositions comprising mutant forms of HPV VLPs, such as HPV VLPs that comprise biologically active fragments and/or mutants of an HPV L1 or L2 protein, including but not necessarily limited to amino acid substitutions, deletions, additions, amino terminal truncations and carboxy-terminal truncations such that these mutations provide for proteins or protein fragments of therapeutic or prophylactic use and would be useful for HPV VLP vaccine development. Any such mutant form of an HPV L1 protein should be capable of forming VLPs and of provoking an immune response against the desired HPV type when administered to a human.

Additionally, one of skill in the art will recognize that the HPV L1 or L1+L2 proteins, which are used to self-assemble VLPs for inclusion in the compositions disclosed herein, may be encoded by a full-length wild-type HPV L1 or L2 polynucleotide, or may be encoded by a fragment or mutant of the known wild-type sequence. Wild-type polynucleotide sequences that encode mRNA expressing HPV L1 or L2 protein are available in the art. Any mutant polynucleotide will encode either a protein or protein fragment which at least substantially mimics the pharmacological properties of an HPV L1 or L2 protein, including the ability to form VLPs that are able to provoke an immune response against the HPV type of interest when administered to a human. Any such polynucleotide includes but is not necessarily limited to: polynucleotides comprising nucleotide substitutions, deletions, additions, amino-terminal truncations and carboxy-terminal truncations.

The amount of virus-like particles of each HPV type to be included in the formulations and compositions of the invention depends on the immunogenicity of the expressed gene product. In general, a therapeutically effective dose of VLPs of any of the at least one HPV type is about 1 μg to about 300 μg. In some embodiments, a therapeutically effective dose of VLPs of any of the at least one HPV type is about 1 μg to about 200 μg. In some embodiments, a therapeutically effective dose of VLPs of any of the at least one HPV type is about 1 μg to about 100 μg. In some embodiments, a therapeutically effective dose of VLPs of any of the at least one HPV type is about 10 μg to about 200 μg. In some embodiments, a therapeutically effective dose of VLPs of any of the at least one HPV type is about 10 μg to about 100 μg. In some embodiments, a therapeutically effective dose of VLPs of any of the at least one HPV type is about 10 μg to about 80 μg. In some embodiments, a therapeutically effective dose of VLPs of any of the at least one HPV type is about 20 μg to about 60 μg.

In some embodiments, a dose of a composition or vaccine includes:
15-160 μg of VLPs of HPV Type 6 L1 protein,
20-200 μg of VLPs of HPV Type 11 L1 protein,
30-280 μg of VLPs of HPV Type 16 L1 protein,
20-200 μg of VLPs of HPV Type 18 L1 protein.
10-120 μg of VLPs of HPV Type 31 L1 protein,
10-120 μg of VLPs of HPV Type 33 L1 protein,
10-120 μg of VLPs of HPV Type 45 L1 protein,
10-120 μg of VLPs of HPV Type 52 L1 protein, and
10-120 μg of VLPs of HPV Type 58 L1 protein.

In some embodiments, a dose of a composition or vaccine includes:
15-120 μg of VLPs of HPV Type 6 L1 protein,
20-150 μg of VLPs of HPV Type 11 L1 protein,
30-210 μg of VLPs of HPV Type 16 L1 protein,
20-150 μg of VLPs of HPV Type 18 L1 protein.
10-90 μg of VLPs of HPV Type 31 L1 protein.
10-90 μg of VLPs of HPV Type 33 L1 protein,
10-90 μg of VLPs of HPV Type 45 L1 protein,
10-90 μg of VLPs of HPV Type 52 L1 protein, and
10-90 μg of VLPs of HPV Type 58 L1 protein.

In some embodiments, a dose of a composition or vaccine includes:
15-80 μg of VLPs of HPV Type 6 L1 protein,
20-100 μg of VLPs of HPV Type 11 L1 protein,
30-140 μg of VLPs of HPV Type 16 L1 protein,
20-100 μg of VLPs of HPV Type 18 L1 protein,
10-60 μg of VLPs of HPV Type 31 L1 protein,
10-60 μg of VLPs of HPV Type 33 L1 protein,
10-60 μg of VLPs of HPV Type 45 L1 protein.
10-60 μg of VLPs of HPV Type 52 L1 protein, and
10-60 μg of VLPs of HPV Type 58 L1 protein.

In some embodiments, a dose of a composition or vaccine includes:
15-40 μg of VLPs of HPV Type 6 L1 protein,
20-50 μg of VLPs of HPV Type 11 L1 protein,
30-70 μg of VLPs of HPV Type 16 L1 protein,
20-50 μg of VLPs of HPV Type 18 L1 protein,
10-30 μg of VLPs of HPV Type 31 L1 protein.
10-30 μg of VLPs of HPV Type 33 L1 protein.
10-30 μg of VLPs of HPV Type 45 L1 protein,
10-30 μg of VLPs of HPV Type 52 L1 protein, and
10-30 μg of VLPs of HPV Type 58 L1 protein.

In some embodiments, a dose of a composition or vaccine includes:
90 μg of VLPs of HPV Type 6 L1 protein.
120 μg of VLPs of HPV Type 11 L1 protein,
180 μg of VLPs of HPV Type 16 L1 protein,
120 μg of VLPs of HPV Type 18 L1 protein,
60 μg of VLPs of HPV Type 31 L1 protein,
60 μg of VLPs of HPV Type 33 L1 protein,
60 μg of VLPs of HPV Type 45 L1 protein,
60 μg of VLPs of HPV Type 52 L1 protein, and
60 μg of VLPs of HPV Type 58 L1 protein.

In some embodiments, a dose of a composition or vaccine includes:
60 μg of VLPs of HPV Type 6 L1 protein,
80 μg of VLPs of HPV Type 11 L1 protein,
120 μg of VLPs of HPV Type 16 L1 protein,
80 μg of VLPs of HPV Type 18 L1 protein,
40 μg of VLPs of HPV Type 31 L1 protein,
40 μg of VLPs of HPV Type 33 L1 protein,
40 μg of VLPs of HPV Type 45 L1 protein,
40 μg of VLPs of HPV Type 52 L1 protein, and
40 μg of VLPs of HPV Type 58 L1 protein.

In some embodiments, a dose of a composition or vaccine includes:
30 μg of VLPs of HPV Type 6 L1 protein,
40 μg of VLPs of HPV Type 11 L1 protein,
60 μg of VLPs of HPV Type 16 L1 protein,
40 μg of VLPs of HPV Type 18 L1 protein,
20 μg of VLPs of HPV Type 31 L1 protein,
20 μg of VLPs of HPV Type 33 L1 protein,
20 μg of VLPs of HPV Type 45 L1 protein,
20 μg of VLPs of HPV Type 52 L1 protein, and
20 μg of VLPs of HPV Type 58 L1 protein.

In some embodiments, the vaccine doses described above, have a total volume of 0.5 mL (i.e., a dose of vaccine composition comprising VLP of HPV types 6, 11, 16, 18, 31, 33, 45, 52, and 58, together with any pharmaceutically acceptable carriers or excipients, have a volume of 0.5 mL).

### The Aluminum Adjuvant

In some embodiments, the compositions of the invention include an ABI and a chitosan or chitosan derivative and further include an aluminum adjuvant. The aluminum adjuvant of the compositions of the invention may include aluminum hydroxide, aluminum phosphate, aluminum hydroxyphosphate, amorphous aluminum hydroxyphosphate sulfate (AAHS) or so-called “alum” (KAl(SO₄)-12H₂O) (see Klein et al., Analysis of aluminum hydroxyphosphate vaccine adjuvants by (27)A1 MAS NMR., J Pharm. Sci. 89(3): 311-21 (2000)). In one embodiment of the invention provided herein, the aluminum adjuvant is aluminum hydroxyphosphate or AAHS. The ratio of phosphate to aluminum in the aluminum adjuvant can range from 0 to 1.3. In some embodiments of this aspect of the invention, the phosphate to aluminum ratio is within the range of 0.1 to 0.70. In some embodiments, the phosphate to aluminum ratio is within the range of 0.2 to 0.50. In some embodiments, the phosphate to aluminum ratio is within the range of 0.7 to 1.2.

One of skill in the art will be able to determine an optimal dosage of aluminum adjuvant that is both safe and effective at increasing the immune response to the targeted HPV type(s). For a discussion of the safety profile of aluminum, as well as amounts of aluminum included in FDA-licensed vaccines, see Baylor et al., Vaccine 20: S18-S23 (2002). In some embodiments, the aluminum adjuvant is present in an amount of about 100 to 3600 μg/dose (200 to 7200 μg/mL concentration). In some embodiments, the aluminum adjuvant is present in an amount of about 100 to 2700 μg/dose (200 to 5400 μg/mL concentration). In some embodiments, the aluminum adjuvant is present in an amount of about 100 to 1800 μg/dose (200 to 3600 μg/mL concentration). In some embodiments, the aluminum adjuvant is present in an amount of about 100 to 900 μg/dose (200 to 1800 μg/mL concentration). In some embodiments of the formulations and compositions of the invention, there is 200-300 μg aluminum adjuvant per dose of vaccine. In alternative embodiments of the formulations and compositions of the invention, there is 300-500 μg aluminum adjuvant per dose of vaccine. In alternative embodiments of the formulations and compositions of the invention, there is 400-1200 μg aluminum adjuvant per dose of vaccine. In alternative embodiments of the formulations and compositions of the invention, there is 1200-2000 μg aluminum adjuvant per dose of vaccine. In some embodiments of the formulations and compositions of the invention, there is equal to or less than 2000 μg aluminum adjuvant per dose of vaccine. In some embodiments of the formulations and compositions of the invention, there is equal to or less than 1500 μg aluminum adjuvant per dose of vaccine. In some embodiments of the formulations and compositions of the invention, there is equal to or less than 1000 μg aluminum adjuvant per dose of vaccine. In some embodiments of the formulations and compositions of the invention, there is equal to or less than 500 μg aluminum adjuvant per dose of vaccine. In some embodiments of the formulations and compositions of the invention, there is equal to or less than 400 μg aluminum adjuvant per dose of vaccine. In some embodiments of the formulations and compositions of the invention, there is equal to or less than 300 μg aluminum adjuvant per dose of vaccine. In some embodiments of the formulations and compositions of the invention, there is equal to or less than 200 μg aluminum adjuvant per dose of vaccine. In some embodiments of the formulations and compositions of the invention, there is equal to or less than 100 μg aluminum adjuvant per dose of vaccine.

### The HPV VLP-Based Vaccine

Any HPV VLP-based vaccine is suitable for use in the pharmaceutical compositions and methods of the invention. Known HPV VLP vaccines can be modified to include both an aluminum adjuvant and a chitosan. New vaccines can be developed according to the invention described herein that comprise at least one HPV type, optionally in the form of an HPV VLP adsorbed to an aluminum adjuvant, in combination with a chitosan. Additionally, new vaccines can be developed according to the invention described herein that comprise at least one HPV type in the form of an HPV VLP adsorbed to an aluminum adjuvant in combination with a chitosan.

One exemplary HPV vaccine is a bivalent vaccine protective against HPV 16 and 18, which is known commercially as CERVARIX® (GlaxoSmithKline Biologicals, Rixensart, Belgium). Another exemplary HPV VLP vaccine is a non-infectious recombinant, quadrivalent vaccine prepared from highly purified VLPs of the major capsid (L1) protein of HPV types 6, 11, 16, and 18, and may be referred to herein by its proprietary name GARDASIL® (Merck & Co., Inc., Rahway, NJ, USA), see Bryan, J. T. Vaccine 25(16): 3001-6 (2007); Shi et al. Clinical Pharmacology and Therapeutics 81(2): 259-64 (2007). Another exemplary HPV VLP vaccine is the nine-valent vaccine approved for prevention of HPV (that includes the capsid (L1) protein of HPV types 6, 11, 16, 18, 31, 33, 45, 52, and 58), which is referred to herein by its proprietary name GARDASIL® 9 (Merck & Co., Inc., Rahway, NJ, USA).

In some embodiments, a vaccine dose includes, in addition to VLPs, an aluminum adjuvant (as amorphous aluminum hydroxyphosphate sulfate), sodium chloride, L-histidine, polysorbate 80, sodium borate, and water. In some embodiments, the HPV vaccine includes 100-3500 μg aluminum adjuvant, 1-50 mg sodium chloride, 0.05-10 mg L-histidine, 1-100 μg polysorbate, 1-100 μg sodium borate, and water. In some embodiments, the HPV vaccine includes about 500 μg aluminum adjuvant, about 9.56 mg sodium chloride, about 0.78 mg L-histidine, about 50 μg polysorbate 80, about 35 μg sodium borate, and water for injection.

In some embodiments of the invention, the pharmaceutical compositions and formulations comprise HPV VLP-based vaccines, or HPV VLPs as described herein, that are monovalent, bivalent, trivalent, quadrivalent, 5-valent, 6-valent, 7-valent, 8-valent or 9-valent. In particular embodiments, the pharmaceutical compositions and formulations are 9-valent. In some embodiments, the pharmaceutical compositions comprise HPV VLP-based vaccines, or HPV VLPs as described herein, with more than four different types of HPV VLPs. For example, the pharmaceutical compositions and formulations of the invention may include HPV VLP-based vaccines, or HPV VLPs as described herein, that are 8-valent, 9-valent, 10-valent, and so forth. In some embodiments, pharmaceutical compositions comprise VLPs of HPV 16 and/or HPV 18, without the inclusion of other HPV VLP types. Multi-valent vaccines comprising different HPV VLPs other than the HPV types included in GARDASIL® or GARDASIL® 9 are also contemplated herein.

In some embodiments, VLPs of HPV types 6 and 11 are included. In some embodiments, VLPs of HPV types 16, 31, and 35 are included. In some embodiments. VLPs of HPV types 18, 45, and 59 are included. In some embodiments, VLPs of HPV types 26, 51, and 69 are included. In some embodiments, VLPs of HPV types 33, 52, and 58 are included. In some embodiments, VLPs of HPV types 39, 68, and 70 are included. In some embodiments, VLPs of HPV types 53, 56, and 66 are included.

In some embodiments, VLPs of HPV types 16 and 18 are included. In some embodiments, VLPs of HPV types 6, 11, 16, and 18 are included. In some embodiments, VLPs of HPV types 6, 18, 52, and 58 are included. In some embodiments, VLPs of HPV types 6, 11, 16, 18, 31, 45, 52, and 58 are included. In some embodiments, VLPs of HPV types 6, 11, 16, 18, 33, 45, 52, and 58 are included. In some embodiments, VLPs of HPV types 6, 11, 16, 18, 31, 33, 45, 52, and 58 are included. In some embodiments, VLPs of HPV types 6, 11, 16, 18, 31, 33, 45, 52, and 59 are included. In some embodiments, VLPs of HPV types 6, 11, 16, 18, 31, 33, 45, 53, and 58 are included. In some embodiments, VLPs of HPV types 6, 11, 16, 18, 31, 33, 45, 53, and 59 are included. In some embodiments, VLPs of HPV types 6, 11, 16, 18, 31, 33, 35, 45, 52, and 58 are included. In some embodiments, VLPs of HPV types 6, 11, 16, 18, 31, 33, 35, 45, 52, 58, and 59 are included. In some embodiments, VLPs of HPV types 6, 11, 16, 18, 31, 33, 45, 52, 58, 59, and 68 are included. In some embodiments, VLPs of HPV types 6, 11, 16, 18, 31, 33, 35, 39, 45, 51, 52, 56, 58, and 59 are included. In some embodiments, VLPs of HPV types 6, 11, 16, 18, 31, 33, 35, 39, 45, 51, 52, 56, 58, 59, and 68 are included. In some embodiments, VLPs of HPV types 6, 11, 16, 18, 31, 33, 35, 39, 45, 51, 52, 56, 58, 59, and 73 are included. In some embodiments. VLPs of HPV types 6, 11, 16, 18, 31, 33, 35, 39, 45, 51, 52, 56, 58, 59, 68 and 73 are included. In some embodiments, VLPs of HPV types 6, 11, 16, 18, 26, 31, 33, 35, 45, 51, 52, 58, 59, and 69 are included. In some embodiments, VLPs of HPV types 6, 11, 16, 18, 26, 31, 33, 35, 39, 45, 51, 52, 58, 59, 68, 69, and 70 are included. In some embodiments. VPs of HPV types 6, 11, 16, 18, 26, 31, 33, 35, 39, 45, 51, 52, 53, 56, 58, 59, 66, 68, 69, and 70 are included.

In some embodiments, the pharmaceutical compositions and formulations comprise HPV VLP-based vaccines and/or antigens as listed in Table I below:

TABLE I

| Name | Antigen | Adjuvant | Company/Institution |
|---|---|---|---|
| CERVARIX ® | L1 VLP of HPV-16 and | Aluminum | GlaxoSmithKline |
| (2vHPV vaccine) | HPV-18 | hydroxide | Biologics (Rixensart, |
| | | and MPL | Belgium) |
| GARDASIL ® | L1 VLP of HPV-6, HPV-11, | AAHS | Merck & Co., Inc., |
| (4vHPV vaccine) | HPV-16 and HPV-18 | | Rahway NJ USA |
| GARDASIL ® 9 | L1 VLP of HPV-6, HPV-11, | AAHS | Merck& Co., Inc., |
| (9vHPV vaccine) | HPV-16, HPV-18, HPV-31, | | Rahway NJ USA |
| | HPV-33, HPV-45, HPV-52 | | |
| | and HPV-58 | | |
| CECOLIN ® | L1 VLP of HPV-16 and | Aluminum | Xiamen Innovax |
| | HPV-18 | hydroxide | |
| GEOCOLIN ® | L1 VLP of HPV-6 and HPV- | Aluminum | Xiamen Innovax |
| | 11 | hydroxide | |
| L1 capsomers | L1 capsomers of HPV-16 | unknown | R. Garcea, University of |
| | | | Colorado- Boulder |
| RG1-VLP | HPV-16 L1-L2 (17-36) VLP | Aluminum | R. Kirnbauer, NCI, |
| | | hydroxide | Pathovax LLC |
| L2-AAV | L2 peptides of HPV-16 and | unknown | 2A Pharma |
| | HPV-31 displayed on AAV | | |
| | VLP | | |
| L2 multimer | Fusion protein of L2~11-88 | Alum | Sanofi, BravoVax |
| | of HPV-6, HPV-16, HPV- | | |
| | 18, HPV-31 and HPV-39 | | |
| L2-thioredoxin | L2 peptide displayed on | unknown | M. Muller, DKFZ |
| | thioredoxin | | |
| AX03 | L2 peptide displayed on | unknown | Agilvax, NIAID |
| | bacteriophage | | |
| L1-E7 VLP | HPV-16 L1-E7 VLP | None | Medigene AG |
| TA-CIN | HPV-16 L2E7E6 fusion | None | Cantab Pharmaceuticals, |
| | protein | | Xenova |
| TA-GW | HPV-6 L2E7 fusion | Aluminum | Cantab Pharmaceuticals, |
| | protein | hydroxide | GSK |
| | | or AS03 | |

In some embodiments, the vaccines of the invention comprise VLPs containing the antigenic determinants required to induce the generation of neutralizing antibodies in the subject. In some embodiments, a chitosan of the invention is combined with a Human Papillomavirus Bivalent (Types 16 and 18) Vaccine, Recombinant. In some embodiments, a chitosan of the invention is combined with CERVARIX®. In some embodiments, a chitosan of the invention is combined with a Human Papillomavirus Quadrivalent (Types 6, 11, 16, 18) Vaccine. Recombinant. In some embodiments, a chitosan adjuvant of the invention is combined with GARDASIL®. In some embodiments, a chitosan of the invention is combined with a Human Papillomavirus 9-valent Vaccine, Recombinant. In some embodiments, a chitosan of the invention is combined with GARDASIL® 9.

In some embodiments, a chitosan or chitosan derivative of the invention is combined with an HPV vaccine.

### Kits of the Invention

Also provided herein are kits including any of the pharmaceutical compositions as described above and instructions for use.

Also provided herein are kits including (a) a vaccine comprising an ABI and a pharmaceutically acceptable carrier and (b) a chitosan or chitosan derivative.

In some embodiments of the kits, the vaccine of (a) comprises an RSV antigen. In some embodiments, the pharmaceutical composition of (a) comprises a recombinant RSV F trimer, wherein the RSV F trimer includes DS-Cav1 substitutions.

In some embodiments of the kits, the vaccine of (a) comprises HPV VLPs of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82. In some embodiments, the vaccine of (a) is an HPV vaccine. In some embodiments, the HPV vaccine is a Human Papillomavirus Bivalent (Types 16 and 18) Vaccine, Recombinant. In some embodiments, the HPV vaccine is CERVARIX®. In some embodiments, the HPV vaccine is a Human Papillomavirus Quadrivalent (Types 6, 11, 16, 18) Vaccine, Recombinant. In some embodiments, the HPV vaccine is GARDASIL®. In some embodiments, the HPV vaccine is a Papillomavirus 9-valent Vaccine, Recombinant. In some embodiments, the HPV vaccine is GARDASIL® 9.

In some embodiments of the kits of the invention, the chitosan or chitosan derivative is any of the chitosan or chitosan derivatives described herein above. In some embodiments, the kit includes 0.1 μg to 100 mg of a chitosan or chitosan derivative. In some embodiments, the kit includes 0.1 μg to 100 mg of a water-soluble chitosan. In some embodiments, the kit includes 0.1 μg to 100 mg of an acid-soluble chitosan. In some embodiments, the kit includes 0.1 μg to 100 mg of trimethyl chitosan.

In some embodiments of the kits of the invention, the kit includes a label or packaging insert that includes a description of the components and/or instructions for use in vivo of the components therein. In some embodiments, the kits include instructions for co-administering (or vaccinating) (a) vaccine and (b) the chitosan or chitosan derivative. In some embodiments, the kits include instructions for admixing (a) the vaccine and (b) the chitosan or chitosan derivative and subsequentially administering (or vaccinating) the admixture to a patient.

### Methods of Treatment of the Invention

Also provided herein is a method of inducing an immune response to an antigen in a human patient comprising co-administering to the patient (a) a pharmaceutical composition comprising an ABI and (b) a chitosan or chitosan derivative. In some embodiments, the chitosan or chitosan derivative is formulated separately from the ABI. In some embodiments, the chitosan or chitosan derivative is formulated with the ABI. In some embodiments, the chitosan or chitosan derivative and ABI are field-mixed to form a composition prior to administration to the patient. In some embodiments, the chitosan or chitosan derivative and ABI are administered sequentially to a patient.

Also provided herein is a method of inducing an immune response to an antigen in a human patient including administering a chitosan or chitosan derivative and an RSV antigen.

Also provided herein is a method of inducing an immune response to an antigen in a human patient including administering a chitosan or chitosan derivative and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82. In some embodiments, the chitosan or chitosan derivative is formulated separately from the VLPs. In some embodiments, the chitosan or chitosan derivative is formulated with the VLPs. In some embodiments, the chitosan or chitosan derivative and VLPs are field-mixed to form a pharmaceutical composition prior to administration to the patient. In some embodiments, the chitosan or chitosan derivative and VLPs are administered sequentially to a patient.

Also provided herein is a method of inducing an immune response to a human papillomavirus (HPV) in a human patient including co-administering to the patient (a) a pharmaceutical composition comprising virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82 and (b) a chitosan or chitosan derivative.

Also provided herein is a method of preventing infection of a human patient by a human papillomavirus (HPV) including administration to the patient a pharmaceutical composition including a chitosan or chitosan derivative and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Also provided herein is a method of inducing a neutralizing titer against an antigen in a subject that includes administering to the subject a pharmaceutical composition including a chitosan or chitosan derivative and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, whereby the administration of the pharmaceutical composition induces a neutralizing titer against the HPV antigen in the subject.

Also provided herein is a method for preventing cancer of a human patient caused by human papillomavirus (HPV) Types 16, 18, 31, 33, 45, 52, and 58 including administration to the patient a pharmaceutical composition including a chitosan or chitosan derivative and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is selected from the group consisting of cervical, vulvar, vaginal, anal, oropharyngeal, and other head and neck cancers.

Also provided herein is a method for preventing cancer of a human patient caused by HPV Types 6, 11, 16, 18, 31, 33, 45, 52, and 58 including administration to the patient a pharmaceutical composition including a chitosan or chitosan derivative and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the cancer is cervical, vulvar, vaginal, or anal precancerous or dysplastic lesions.

Also provided herein is a method for preventing a pathological condition of a human patient caused by HPV Types 6 and 11 including administration to the patient a pharmaceutical composition including a chitosan or chitosan derivative and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the pathological condition is genital warts or condyloma acuminata.

Also provided herein is a method for preventing precancerous or dysplastic lesions of a human patient caused by HPV Types 6, 11, 16, 18, 26, 31, 33, 35, 39, 45, 51, 52, 53, 55, 56, 58, 59, 66, 68, 73, and 82 including administration to the patient a pharmaceutical composition including a chitosan or chitosan derivative and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82, wherein the lesions are selected from cervical intraepithelial neoplasia (CIN) grade 2/3, cervical adenocarcinoma in situ (AIS), cervical intraepithelial neoplasia (CIN) grade 1, vulvar intraepithelial neoplasia (VIN) grade 2 and grade 3, vaginal intraepithelial neoplasia (VaIN) grade 2 and grade 3, anal intraepithelial neoplasia (AIN) grades 1, 2, and 3.

Also provided herein is a method for preventing HPV-related anogenital disease of a human patient caused by HPV Types selected from the group consisting of HPV types: 6, 11, 16, 18, 26, 31, 33, 35, 39, 45, 51, 52, 53, 55, 56, 58, 59, 66, 68, 73, and 82 including administration to the patient a pharmaceutical composition including a chitosan or chitosan derivative and virus-like particles (VLPs) of at least one type of human papillomavirus (HPV) selected from the group consisting of HPV types: 6, 11, 16, 18, 26, 31, 33, 35, 39, 45, 51, 52, 53, 55, 56, 58, 59, 66, 68, 73, and 82.

Embodiments of the invention that include an ABI directed against HPV also include one or more of the pharmaceutical compositions described herein (i) for use in, (ii) for use as a medicament or composition for, or (iii) for use in the preparation of a medicament for: (a) therapy (e.g., of the human body); (b) medicine; (c) induction of an immune response against HPV types included in the vaccine (d) decreasing the likelihood of HPV infection in a patient; (e) prevention of infection with HPV types in the vaccine, (f) prevention or reduction of the likelihood of cervical cancer, (g) prevention or reduction of the likelihood of vulvar cancer, (h) prevention or reduction of the likelihood of vaginal cancer, (i) prevention or reduction of the likelihood of anal cancer, (j) prevention or reduction of the likelihood of oropharyngeal cancer, (k) prevention or reduction of the likelihood of other head and neck cancers. (k) prevention or reduction of the likelihood of precancerous or dysplastic anal lesions, (l) prevention or reduction of the likelihood of genital warts or condyloma acuminata, (m) prevention or reduction of the likelihood of cervical intraepithelial neoplasia (CIN) grade 2/3 lesions, (n) prevention or reduction of the likelihood of cervical adenocarcinoma in situ (AIS) lesions, (o) prevention or reduction of the likelihood of cervical intraepithelial neoplasia (CIN) grade 1 lesions, (p) prevention or reduction of the likelihood of vulvar intraepithelial neoplasia (VIN) grade 2 and grade 3 lesions, (q) prevention or reduction of the likelihood of vaginal intraepithelial neoplasia (VaIN) grade 2 and grade 3 lesions, (r) prevention or reduction of the likelihood of anal intraepithelial neoplasia (AIN) grades 1, 2, and 3 lesions.

Embodiments of the invention that include an ABI directed against RSV also include one or more of the pharmaceutical compositions described herein (i) for use in, (ii) for use as a medicament or composition for, or (iii) for use in the preparation of a medicament for: (a) therapy (e.g., of the human body); (b) medicine; (c) induction of an immune response against RSV antigens included in the vaccine (d) decreasing the likelihood of RSV infection in a patient; (e) prevention of infection of RSV.

In embodiment 1, the invention provides a pharmaceutical composition comprising an active biological ingredient (ABI), a chitosan or a chitosan derivative, and a pharmaceutically acceptable carrier.

In embodiment 2, the pharmaceutical composition of embodiment 1 is provided, wherein the pharmaceutical composition is made by mixing a vaccine and a chitosan or a chitosan derivative; wherein the vaccine comprises the ABI and a pharmaceutically acceptable carrier.

In embodiment 3, the pharmaceutical composition of either of embodiments 1-2 is provided, wherein the chitosan comprises an acid soluble chitosan.

In embodiment 4, the pharmaceutical composition of either of embodiments 1-2 is provided, wherein the chitosan comprises a water-soluble chitosan.

In embodiment 5, the pharmaceutical composition of any of embodiments 1-4 is provided, wherein the chitosan has a molecular weight of 5 kDa to 100 kDa.

In embodiment 6, the pharmaceutical composition of any of embodiments 1-5 is provided, wherein the chitosan has a viscosity of 1 cP at 20° C. to about 125 cP at 20° C., when measured with a viscometer at 20° C. at a standard concentration.

In embodiment 7, the pharmaceutical composition of any of embodiments 1-2 is provided, wherein the chitosan derivative comprises a trimethyl chitosan.

In embodiment 8, the pharmaceutical composition of any of embodiments 1-2 and 7 is provided, wherein the chitosan derivative has a molecular weight greater than 100 kDa.

In embodiment 9, the pharmaceutical composition of any of embodiments 1-2 and 7-8 is provided, wherein the chitosan derivative has a viscosity of about 5 cP at 20° C. to about 30 cP at 20° C., when measured with a viscometer at 20° C. at a standard concentration.

In embodiment 10, the pharmaceutical composition of any of embodiments 1-2 and 7-9 is provided, wherein the pharmaceutical composition comprises trimethyl chitosan having an average molecular weight of between about 225 kDa and about 275 kDa.

In embodiment 11, the pharmaceutical composition of any of embodiments 1-2 and 7-9 is provided, wherein the pharmaceutical composition comprises trimethyl chitosan having an average molecular weight of between about 125 kDa and about 160 kDa.

In embodiment 12, the pharmaceutical composition of any of embodiments 1-2 and 7-9 is provided, wherein the pharmaceutical composition comprises trimethyl chitosan having an average molecular weight of between about 100 kDa and about 150 kDa.

In embodiment 13, the pharmaceutical composition of any of embodiment 1-2, 7-9, and 10 is provided, wherein the pharmaceutical composition comprises trimethyl chitosan having an average molecular weight of between about 225 kDa and about 275 kDa and a degree of about quaternization of 30-70%.

In embodiment 14, the pharmaceutical composition of any of embodiments 1-2, 7-9, and 11 is provided, wherein the pharmaceutical composition comprises trimethyl chitosan having an average molecular weight of between about 125 kDa and about 160 kDa and a degree of about quaternization of 40-60%.

In embodiment 15, the pharmaceutical composition of any of embodiments 1-2, 7-9, and 12 is provided, wherein the pharmaceutical composition comprises trimethyl chitosan having an average molecular weight of between about 100 kDa and about 150 kDa and a degree of about quaternization of greater than 50%.

In embodiment 16, the pharmaceutical composition of any of embodiments 1-15 is provided wherein the pharmaceutical composition further comprises a salt.

In embodiment 17, the pharmaceutical composition of any of embodiments 1-16 is provided, wherein the pharmaceutical composition further comprises a buffer present in an amount of about 1 mMol to about 100 mMol, wherein the buffer is selected from the group consisting of: acetic acid, histidine, citrate, Bis-Tris, HEPES, phosphate, MES, sodium chloride, and combinations thereof.

In embodiment 18, the pharmaceutical composition of any of embodiments 1 to 17 is provided wherein the pharmaceutical composition further comprises a tonicity modifier present in an amount of about 10 mMol to about 50 mMol, wherein the tonicity modifier is selected from the group consisting of: sodium chloride, potassium chloride, sucrose, trehalose and combinations thereof.

In embodiment 19, the pharmaceutical composition of any of embodiments 1-18 is provided, wherein the pharmaceutical composition further comprises a detergent present in an amount of about 0.001% (w/v) to about 0.2% (w/v), wherein the detergent is selected from the group consisting of Polysorbate 80, Polysorbate 20, Poloxamer 188, and combinations thereof.

In embodiment 20, the pharmaceutical composition of any of embodiments 1-19 is provided, wherein the pharmaceutical composition further comprises an aluminum adjuvant.

In embodiment 21, the pharmaceutical composition of any of embodiments 1-19 is provided, wherein the ABI is a recombinant syncytial virus (RSV) antigen.

In embodiment 22, the pharmaceutical composition of any of embodiments 1-21 is provided, wherein the ABI comprises a recombinant RSV F trimer, wherein the RSV F trimer includes DS-Cav1 substitutions.

In embodiment 23, the pharmaceutical composition of any of embodiments 1-20 is provided, wherein the ABI comprises virus-like particles (VLPs) of human papillomavirus.

In embodiment 24, the pharmaceutical composition of embodiment 23 is provided, wherein composition comprises HPV VLPs of at least one HPV type selected from the group consisting of HPV type 6, 11, 16, 18, 26, 31, 33, 35, 39, 45, 51, 52, 53, 55, 56, 58, 59, 66, 68, 69, 70, 73, and 82.

In embodiment 25, the pharmaceutical composition of embodiment 24 is provided, wherein composition comprises HPV VLPs of HPV types 16 and 18.

In embodiment 26, the pharmaceutical composition of embodiment 25 is provided, wherein the composition further comprises HPV VLPs of HPV types 6 and 11.

In embodiment 27, the pharmaceutical composition of any of embodiments 25-26 is provided, wherein composition comprises further comprises HPV VLPs of HPV types 31, 33, 45, 52 and 58.

In embodiment 28, the pharmaceutical composition of embodiment 24 is provided, wherein the ABI comprises VLPs of HPV types 6, 11, 16, 18, 31, 33, 35, 39, 45, 51, 52, 56, 58, and 59.

In embodiment 29, the pharmaceutical composition of any of embodiments 23-28 is provided, wherein the HPV VLPs comprise recombinant HPV L1 or recombinant HPV L1+L2 protein.

In embodiment 30, the pharmaceutical composition of any of embodiments 24-28 is provided, wherein the HPV VLPs of each of the at least one HPV types are present in a concentration of about 10 μg to about 300 μg per 0.5 mL of the pharmaceutical composition, and wherein the total HPV VLP concentration is between 10 μg and 2000 μg per 0.5 mL of the pharmaceutical composition.

In embodiment 31, the pharmaceutical composition of any of embodiments 23-28 is provided, wherein the HPV VLPs comprise HPV L1 protein and do not comprise HPV L2 protein.

In embodiment 32, a method of inducing an immune response to an antigen in a human patient is provided comprising administering to the patient the pharmaceutical composition of any of embodiments 1-27.

In embodiment 33, a method of inducing an immune response to an antigen in a human patient is provided comprising co-administering to the patient (a) a pharmaceutical composition comprising an active biological ingredient (ABI) and (b) a chitosan or chitosan derivative.

In embodiment 34, a method of preventing infection of a human patient is provided comprising administration to the patient the pharmaceutical composition of any of embodiments 1-27.

In embodiment 35, a kit is provided comprising: (a) a vaccine comprising an active biological ingredient (ABI) and a pharmaceutically acceptable carrier; and (b) a chitosan or a chitosan derivative.

In embodiment 36, the kit of embodiment 31 is provided further comprising instructions for administering to a human patient the vaccine and the chitosan or chitosan derivative.

In embodiment 37, a method of inducing a neutralizing titer against an antigen in a patient is provided comprising:
administering to the patient a pharmaceutical composition comprising:
   a chitosan or chitosan derivative, and
   an active biological ingredient (ABI),
   whereby the administration of the pharmaceutical composition induces a neutralizing titer against the antigen in the patient.

### EXAMPLES

### Example 1: Preparation of 80 kDa MW Water-Soluble Chitosan

In this example, water soluble chitosan at 80 kDa was prepared at 22.5 mg/mL concentrations of free chitosan in a 10 mM histidine buffer at a pH of ~5.7. First, 50 mL of the water soluble 80 kDa chitosan was prepared by first placing approximately 40 mL of water into a 100 mL glass beaker. Next, the impeller of an overhead mixer was placed in the beaker and stirred at a modest rate of speed. 0.775 g of histidine (final conc of 10 mM) was weighed out, added to the beaker of water, and the combination was mixed until all solids were dissolved. Then, 1.330 g of chitosan was weighed out, added to the beaker, and stirred until all solids were dissolved. The solution was QS'd to 50 mL using a volumetric flask and sterile filtered in a biosafety cabinet, using a 0.8/0.2 um Supor Membrane Pall Acrodisc syringe filter.

### Example 2: Preparation of 20 kDa MW Water-Soluble Chitosan

In this example, water soluble chitosan at 20 kDa was prepared at 22.5 mg/mL concentrations of free chitosan in a 10 mM histidine buffer at a pH of ~5.7. First, 50 mL of the water soluble 20 kDa chitosan was prepared by first placing approximately 40 mL of water into a 100 mL glass beaker. Next the impeller of an overhead mixer was placed in the beaker and stirred at a modest rate of speed. 0.775 g of histidine (final conc of 10 mM) was weighed and added to the beaker of water, and mixed until all solids were dissolved. 1.3235 g of chitosan was weighed, added to the beaker, and stirred until all solids were dissolved. The solution was then QS'd to 50 mL using a volumetric flask and sterile filtered in a biosafety cabinet, using a 0.8/0.2 um Supor Membrane Pall Acrodisc syringe filter.

### Example 3: Preparation of 20 kDa MW Acid-Soluble Chitosan

In this example, an acid soluble chitosan at 20 kDa was prepared at 22.5 mg/mL concentrations of free chitosan in a 10 mM histidine buffer at a pH of ~5.7. First, 50 mL of the acid soluble 20 kDa chitosan was prepared by first placing approximately 40 mL of a 1% acetic acid solution into a 10 mL glass beaker. Next, the impeller of an overhead mixer was placed in the beaker and stirred at a modest rate of speed. 1.142 g of chitosan was weighed, added to the beaker, and stirred until all solids were dissolved. 0.775 g of histidine (final conc of 10 mM) was weighed, added to the beaker of water, and mixed until all solids were dissolved. The solution was then QS'd to 50 mL using a volumetric flask and sterile filtered in a biosafety cabinet, using a 0.8/0.2 um Supor Membrane Pall Acrodisc syringe filter.

### Example 4: Viscosity Vs Molecular Weight

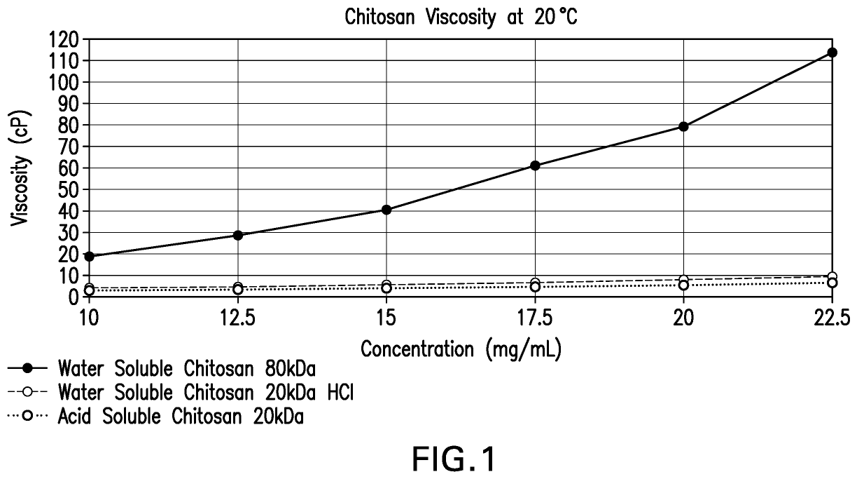

Viscosity for each chitosan solution described in Examples 1, 2, and 3 was measured at 20° C. using a Brookfield DVII+pro viscometer at 20° C., over a range of concentrations. It was surprisingly found that a change in MW of 4-fold resulted in a greater than 10-fold change in viscosity. The results of the viscosity measurements can be found below in Table II and a visual representation is provided in FIG. 1.

TABLE II
Viscosity of solutions of chitosan of different
average molecular weight and concentration.

| Water Soluble Chitosan | | Acid Soluble Chitosan | | Water Soluble Chitosan | |
|---|---|---|---|---|---|
| MW = 80 kDa | | MW = 20 kDa | | MW = 20 kDa | |
| concentration | viscosity(cP) | concentration | viscosity | concentration | viscosity(cP) |
| (mg/mL) | at 20° C. | (mg/mL) | (cP) at 20° C. | (mg/mL) | at 20° C. |
| 22.5 | 113.64 | 22.5 | 9.46 | 22.5 | 6.57 |
| 20 | 79.28 | 20 | 7.99 | 20 | 5.37 |
| 17.5 | 61.01 | 17.5 | 6.65 | 17.5 | 4.75 |
| 15 | 40.5 | 15 | 5.56 | 15 | 4.02 |
| 12.5 | 28.6 | 12.5 | 4.59 | 12.5 | 3.45 |
| 10 | 18.79 | 10 | 3.72 | 10 | 2.95 |

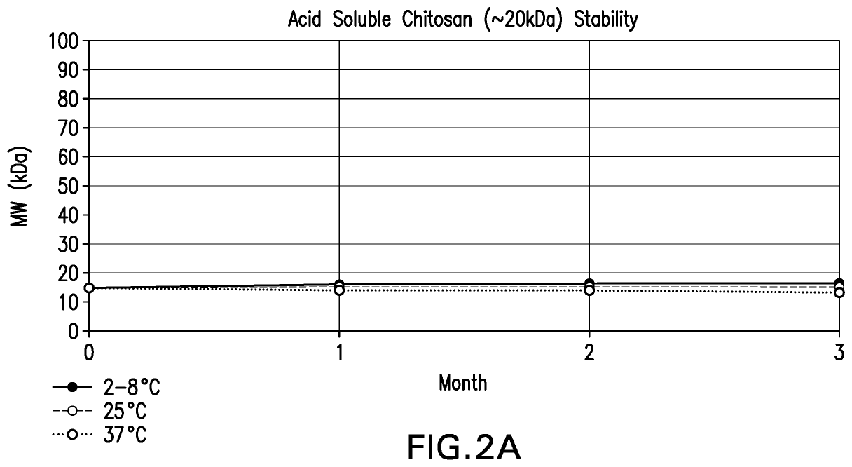

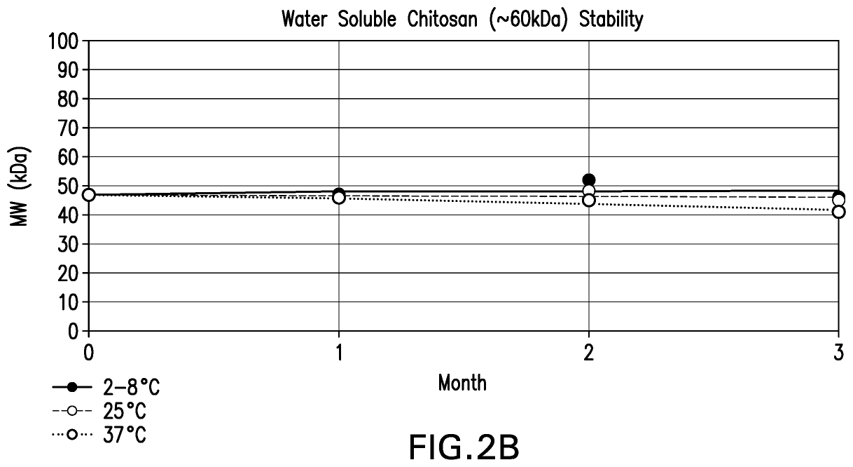

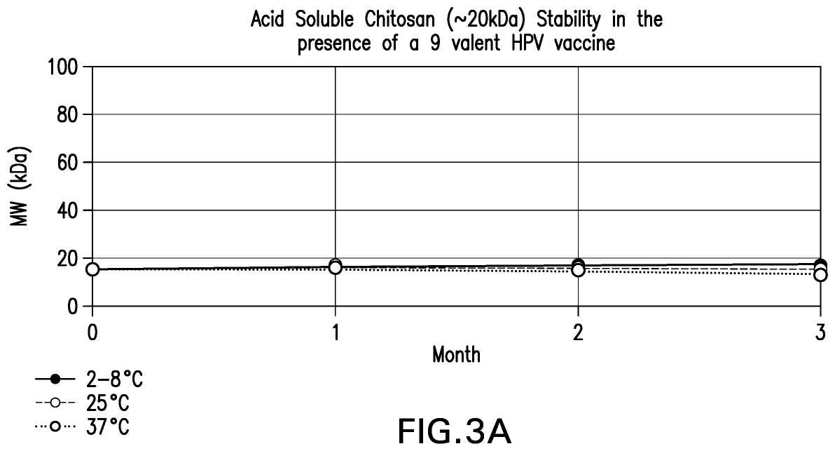

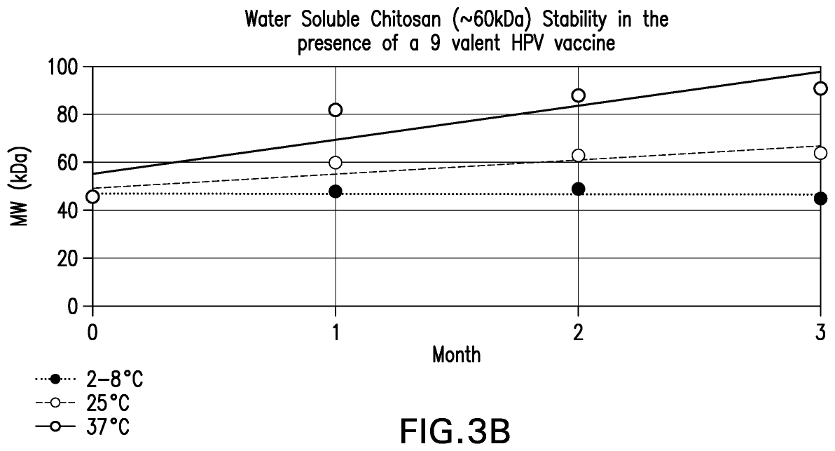

Solutions of water soluble (60 kDa) chitosan and acid soluble (20 kDa) chitosan were prepared as described above, and these were subjected to thermal stresses of up to 37° C. for up to 3 months and stability was monitored by looking for changes in average molecular weight. Samples from these studies were analyzed by HPSEC-MALS-RI to measure average molecular weight. When stability of the chitosan in buffer alone was assessed, a similar stability was observed for both molecular weights tested (FIG. 2). In contrast, when the chitosan solutions were combined with a 9 valent HPV/aluminum adjuvant vaccine that included the capsid (L1) protein of HPV types 6, 11, 16, 18, 31, 33, 45, 52, and 58 (hereinafter “9vHPV Vaccine”) and further contained aluminum (provided as AAHS), sodium chloride, L-histidine, and polysorbate 80) and subjected to similar thermal stresses, a dramatically different stability profile was observed (FIG. 3). Prior to sample analysis the 9 valent HPV vaccine/aluminum adjuvant components were removed from the samples using low speed centrifugation to pellet the aluminum adjuvant and associated VLPs. The supernatant (containing chitosan but not aluminum adjuvant with bound VLPs) was then analyzed by HPSEC-MALS-RI to measure average molecular weight. The lower MW chitosan had significantly better stability (no aggregation of chitosan observed over time) than the higher MW chitosan in the presence of the 9 valent HPV vaccine. This unexpected advantage of the lower molecular weight chitosan would not have been predicted as both are linear polymers of the same polysaccharide, and thus would be expected to have similar stability.

### Example 5: Examining Chitosan Viscosity, Average Molecular Weight, and Concentration

Significant variability in the viscosity of commercially sourced chitosan or chitosan hydrochloride samples from batch to batch (even of the same catalog order number) was observed. Accordingly, it was desired to determine the impact of molecular weight and concentration on the viscosity of chitosan solutions.

To conduct such studies, chitosans of different average molecular weights were analyzed. These were obtained through generating a series, or ladder, of different average molecular weight chitosan samples by enzymatic cleavage of chitosan starting material (average molecular weight from about 60 kDa to about 85 kDa) for different lengths of time. Some commercially available sources of different lower molecular weight chitosans were also identified and sourced directly.

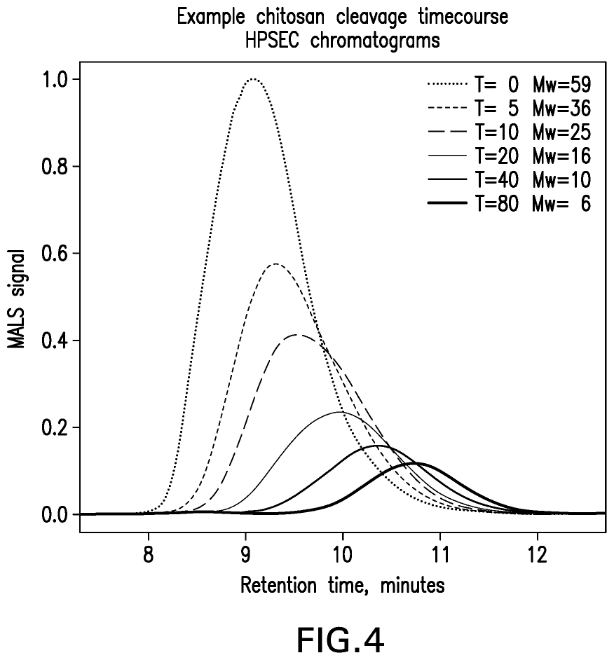

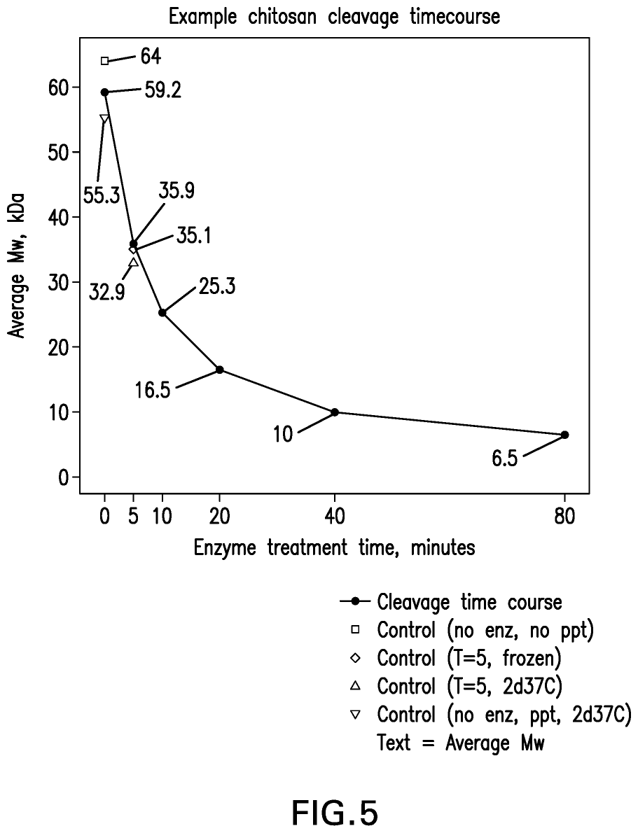

FIGS. 4 and 5 illustrate the chitosan cleavage process. A starting sample of chitosan hydrochloride (high molecular weight (HMC), Germany) with an average molecular weight (measured by HPSEC-MALS-RI) of about 60 kDa, was dissolved in a dilute aqueous acetate buffer and incubated with a small amount of chitosanase enzyme (chitosanase, Streptomyces sp. N174, Millipore-Sigma product 220477-M) at 25° C. for different lengths of time. Aliquots of the incubating chitosan sample were removed at different timepoints (5, 10, 20, 40 and 80 minutes). To quench the enzymatic cleavage reaction, a small volume of 5N sodium hydroxide was added to the timepoint samples, enough to result in the precipitation of chitosan and inactivation of the chitosanase. Pellets were washed multiple times with water and redissolved in dilute acetate buffer, pH 5. HPSEC-MALS-RI was performed to measure the average molecular weight of the cleavage time course samples. FIG. 4 shows overlaid HPSEC chromatograms of chitosan sampled at different cleavage timepoints. FIG. 5 shows the average molecular weight of samples from a cleavage time course, along with several control samples that were tested to demonstrate suitability of our procedure.

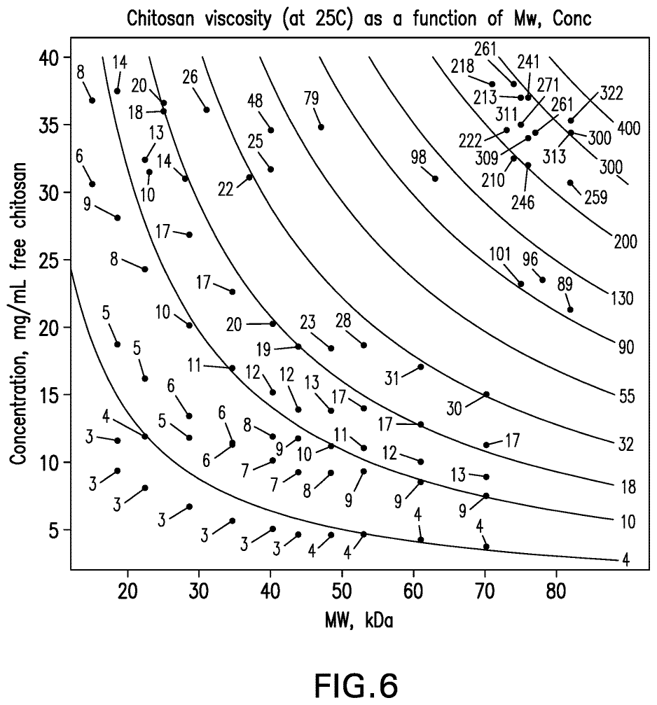

Many chitosan samples were prepared using different batches of starting chitosan, quenching the enzymatic cleavage reaction with addition of base or in some cases by using a heat treatment procedure in place of base precipitation to stop the rapid chitosan enzymatic cleavage reaction. Samples from these studies were analyzed by HPSEC-MALS-RI to measure average molecular weight and concentration, and the viscosity of the samples was also measured. From these data, an empirical model to predict viscosity as a function of concentration and average molecular weight was developed. FIG. 6 shows a plot of the average molecular weight and concentration of samples, with their actual measured viscosity, overlaid on a contour plot depicting the empirical model. It was surprisingly found that there is a strong non-linear dependence of chitosan sample viscosity on both concentration and molecular weight. Not wishing to be bound by theory, the empirical model that was selected based on this data, having desirable statistical and mathematical properties, is shown in Table 11. Other equations may also adequately model the viscosity/molecular weight/concentration relationship, and differences in process or formulation may have impacts that cause deviation from this empirical model.

TABLE III
An empirical model for the viscosity of chitosan samples
as a function of molecular weight and concentration.
lm(formula = log(visc)~I((log(mw)) * (log(conc))) + log(conc) +
I((log(conc))\hat{ }2) + log(mw) + I((log(mw))\hat{ }2), data = df)
Residuals:

| Min | 1Q | Median | 3Q | Max |
|---|---|---|---|---|
| −0.40506 | −0.05271 | 0.01134 | 0.06323 | 0.26988 |

Coefficients:

| | Estimate | Std. Error | t value | Pr(>\|t\|) |
|---|---|---|---|---|
| (Intercept) | 9.14057 | 1.08437 | 8.429 | 2.00e−12 *** |
| I((log(mw)) * | 0.79755 | 0.04973 | 16.038 | <2e−16 *** |
| (log(conc))) | | | | |
| log(conc) | −3.18231 | 0.29056 | −10.952 | <2e−16 *** |
| I((log(conc))\hat{ }2) | 0.31050 | 0.03386 | 9.169 | 7.97e−14 *** |
| log(mw) | −4.05010 | 0.51055 | −7.933 | 1.73e−11 *** |
| I((log(mw))\hat{ }2) | 0.48213 | 0.06732 | 7.162 | 4.89e−10 *** |

Signif. codes: 0
‘***’ 0.001
‘**’ 0.01
‘*’ 0.05
‘.’ 0.1
‘ ’ 1

Residual standard error: 0.1179 on 74 degrees of freedom
Multiple R-squared: 0.9941, Adjusted R-squared: 0.9937
F-statistic: 2489 on 5 and 74 DF, p-value: <2.2e-16

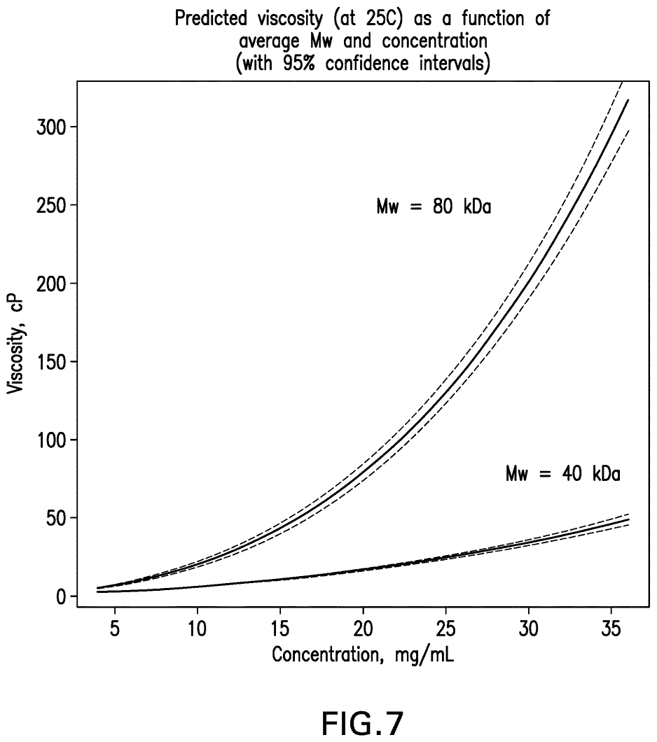

As a further illustration, FIG. 7 shows model predictions for viscosity as a function of concentration for two average molecular weights (80 kDa and 40 kDa). The high viscosities of the 80 kDa sample would pose significant challenges for process, analytical, formulation and filling activities. Recognizing the advantages and conveniences of lower viscosity samples in a manufacturing setting, these studies compared the adjuvant activity of different average molecular weight chitosan samples, described in Example 6, and advantageously found lower molecular weight, less viscous chitosan samples to retain significant adjuvant activity (see Example 6).

### Example 6: Lower Molecular Weight Chitosan Samples Retain Adjuvant Activity and Gain the Advantage of Lower Viscosity

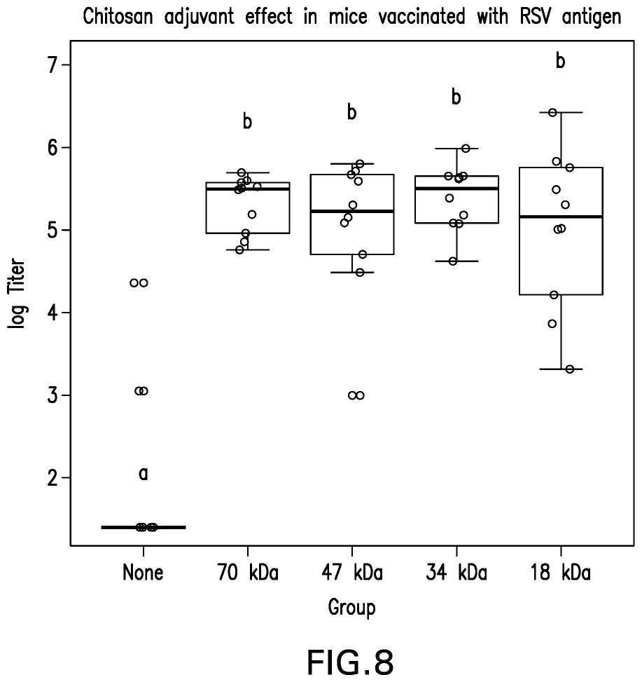

Chitosan samples for which the average molecular weight had been reduced by enzymatic cleavage, prepared as described in Example 5, were used to prepare vaccines for a mouse immunization study with an RSV antigen. The study involved 10 mice per group, and each mouse received 2 vaccinations, given three weeks apart (second dose on or about day 21). To assess immunogenicity and adjuvant activity, total IgG ELISA was measured at week 10 (on or about day 70). As shown in FIG. 8, the starting chitosan and size-reduced chitosan samples show adjuvant activity (measured by total IgG ELISA) that is comparable in a mouse immunogenicity study with an RSV antigen and 50 mcg chitosan per dose.

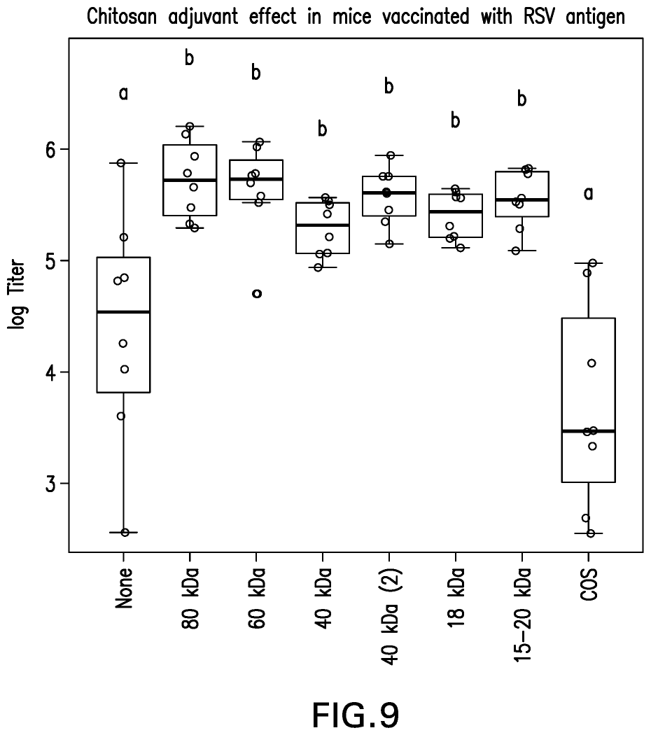

A second study was performed to confirm this observation, using chitosan starting materials from different lots, a small, cleaved chitosan sample from Example 5, additional chitosans of low molecular weight from various suppliers, and extensively size reduced chitosan oligosaccharide (COS). The result was again confirmed that over the range of about 80 kDa to about 15 kDa average molecular weight, adjuvant activity in mice immunized with RSV antigen was present and comparable (FIG. 9). It was noted that the adjuvant activity of the chitosan oligosaccharide was reduced in this study. A preferred range therefore may be chitosan above at least about 15 kDa. Further studies may be conducted to test the adjuvant activity of samples with average molecular weights between about 15 kDa and the size of chitosan oligosaccharide (less than about 2 kDa).

### Example 7: Comparison of the Solubility and Viscosity of Chitosan and Chitosan Derivative Samples

Samples of chitosan derivatives were analyzed to assess solubility and viscosity. The first chitosan derivative analyzed was a low MW TMC, supplied by Millipore Sigma (product 912700). This low MW TMC was tested in 10 different solutions, some of which were at and above pH 7, a pH condition at which chitosan is not soluble. LMW trimethyl chitosan solid was weighed and dispensed into glass vials. The different solutions (as described below in Table IV) were added to the trimethyl chitosan containing vials (one solution per vial), to result in concentrations of approximately about 30 mg/mL trimethyl chitosan. In every case, the trimethyl chitosan samples dissolved, and appeared to result in samples with an observed lower viscosity than had been observed previously for chitosan solutions that included chitosan having similar molecular weights or below. Table IV shows the 10 solutions tested, with the initial and final measured pH values.

TABLE IV
Trimethyl chitosan solubility test solutions,
with pH measured before and after dissolution.

| | pH before | pH after dissolving |
|---|---|---|
| | adding to | trimethyl chitosan |
| | trimethyl | to approximately |
| Solubility Test Solution | chitosan | 30 mg/mL |
| Water alone | pH not | 6.1 |
| | measured | |
| 0.9% sodium chloride | 5.0 | 6.3 |
| 30 mM sodium acetate | 5.0 | 5.5 |
| 50 mM histidine | 5.8 | 5.8 |
| 0.5M NaCl, 20 mM histidine, | 6.2 | 6.3 |
| Phosphate buffered saline | 7.1 | 6.4 |
| 30 mM HEPES | 7.5 | 6.6 |
| 30 mM Tris / Tris HCl | 7.0 | 6.2 |
| 30 mM Tris / Tris HCl | 7.6 | 6.5 |
| 30 mM Tris / Tris HCl | 8.1 | 7.0 |

To investigate the apparent lower viscosity of trimethyl chitosan, samples were prepared of “low molecular weight” (LMW, Millipore Sigma product 912700), “medium molecular weight” (MMW, Millipore Sigma product 912123) and “high molecular weight” (HMW, Millipore Sigma product 912034) trimethyl chitosan at different relatively high concentrations, dissolved in water.

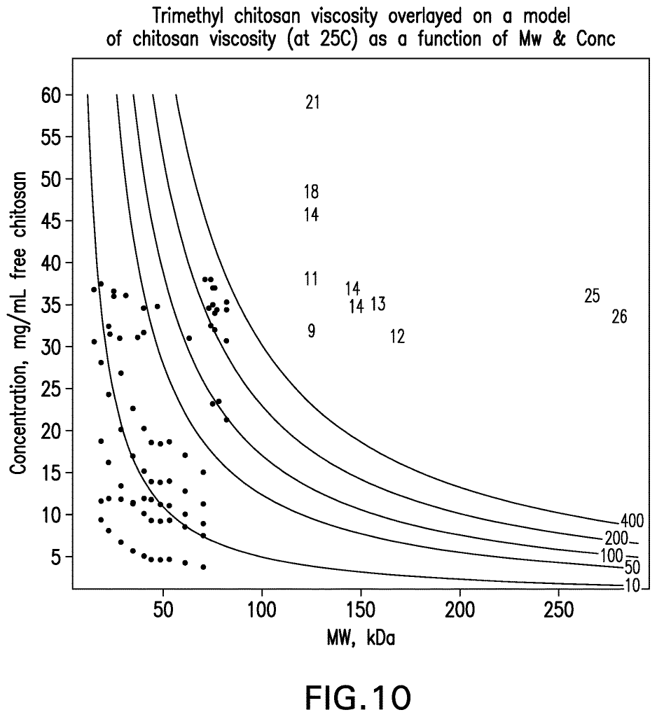

After dissolving the trimethyl chitosan samples in water, the concentration and molecular weight were measured using HPSEC-MALS-RI. The viscosity of the samples was measured using a microVisc capillary viscometer (Rheosense, California, USA), at a temperature of about 20-25 C. As shown on FIG. 10, the value of the viscosity (as numeric text positioned at the appropriate concentration on the y-axis and molecular weight on the x-axis) was overlaid on a plot of the chitosan viscosity model described in Example 5 and shown in FIG. 6. The highest chitosan contour in the model shown is 400 cp. The viscosity of the trimethyl chitosan samples appeared to behave very differently than chitosan. Specifically, as shown in FIG. 10, it was surprisingly found that the viscosity of any of the three trimethyl chitosan samples tested was in the range of about 9-26 cp. In particular, as evidenced by the numbers on the graph, the viscosity of the low molecular weight TMC was measured to be approximately 9 cp, 11 cp, 14 cp, 18 cp, and 21 cp, the viscosity of the medium molecular weight TMC was measured to be approximately 12 cp, 13 cp, and 14 cp; and the high molecular weight TMC was measured to be approximately 25 cp and 26 cp. This surprising result amounted to a viscosity of at least 10 times less than was expected from the model for chitosan samples in the same range of concentration and molecular weight.

### Example 8: Comparative Study of the Stability (at Different Temperatures Over Time) of the Viscosity and Molecular Weight of Samples of Chitosan or Trimethyl Chitosan

A sample of chitosan was prepared by dissolution of 80 kDa chitosan hydrochloride in dilute histidine buffer. Samples of “low molecular weight” (LMW, Millipore Sigma product 912700), “medium molecular weight” (MMW, Millipore Sigma product 912123) and “high molecular weight” (HMW, Millipore Sigma product 912034) trimethyl chitosan were dissolved in water. All samples were sterile filtered (0.22 micron).

The above four samples were aliquoted into glass vials, stoppered and capped. Vials were placed at different temperatures (2-8 C. 25 C. 37 C, and 45 C) for time periods of up to 3 months. At planned stability timepoints, samples were removed from incubators and placed at −70 C. When all samples were available at the end of the stability time course, frozen samples were thawed and prepared for HPSEC-MALS-RI analysis to measure concentration and molecular weight of the chitosan or trimethyl chitosan. This was done by diluting the samples to an estimated 0.5 mg/mL in an acetic acid/sodium acetate buffer (pH 4.5), and diluted samples were frozen at −70 C until HPSEC analysis. Aliquots of the undiluted samples were prepared for viscosity measurements, and for animal immunogenicity studies.

HPSEC-MALS-RI analysis was performed on the samples to measure the (pre-dilution) concentration of the samples (calculated from the measured concentration of the diluted samples and the known dilution factor) and the average molecular weight of the samples. The dn/dc value used for chitosan was 0.190 mL/g, and the dn/dc used for trimethyl chitosan was 0.145 mL/g. Table V lists the anticipated concentrations of the samples (based on a preliminary HPSEC measurements) and the average concentrations for the samples as measured in this study.

TABLE V
Average molecular weights and concentrations of
chitosan and trimethyl chitosan samples used
in the thermal stability study in Example 8.

| | Average | | Average |
|---|---|---|---|
| | initial | Anticipated | measured |
| | molecular | (preliminary) | concentration |
| | weight | concentration | in this study |
| Chitosan control | 80 kDa | 21.7 mg/mL | 24 mg/mL |
| (from chitosan | | | |
| hydrochloride) | | | |
| “low molecular | 116 kDa | 21.5 mg/mL | 27 mg/mL |
| weight” trimethyl | | | |
| chitosan | | | |
| “medium molecular | 147 kDa | 35.2 mg/mL | 37 mg/mL |
| weight” trimethyl | | | |
| chitosan | | | |
| “high molecular | 260 kDa | 35.5 mg/mL | 39 mg/mL |
| weight” trimethyl | | | |
| chitosan | | | |

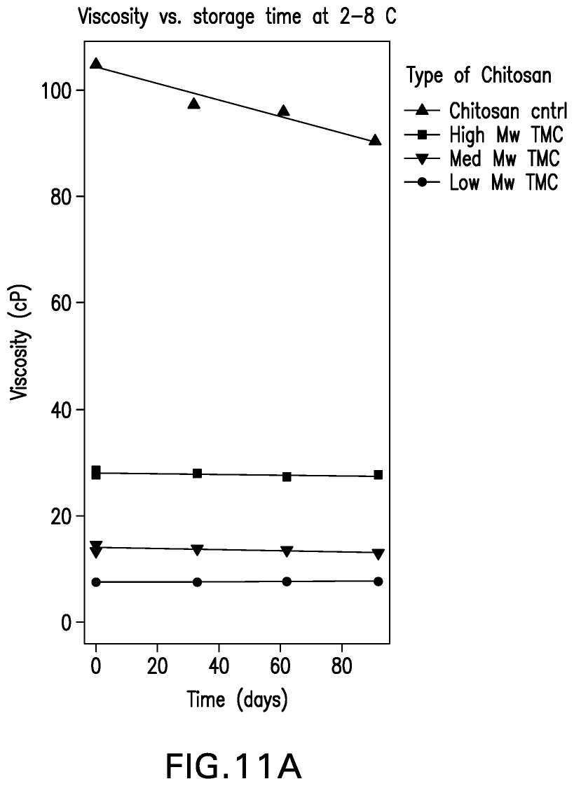

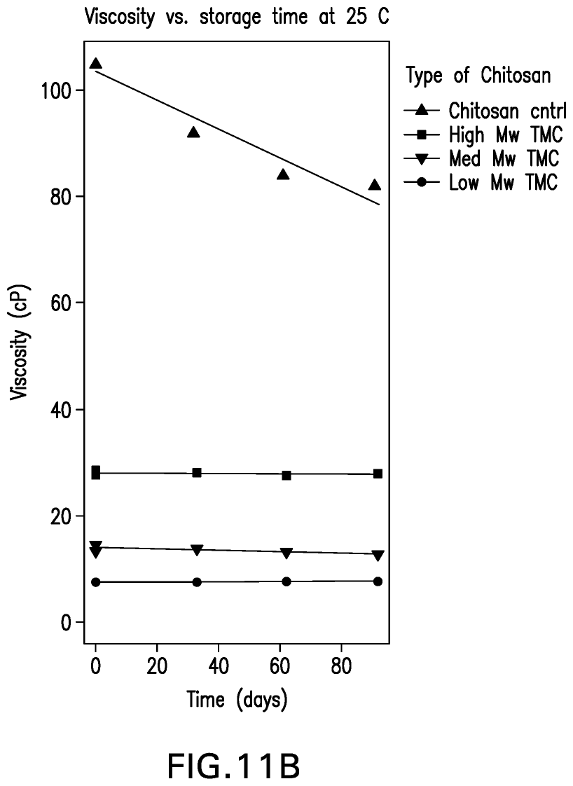

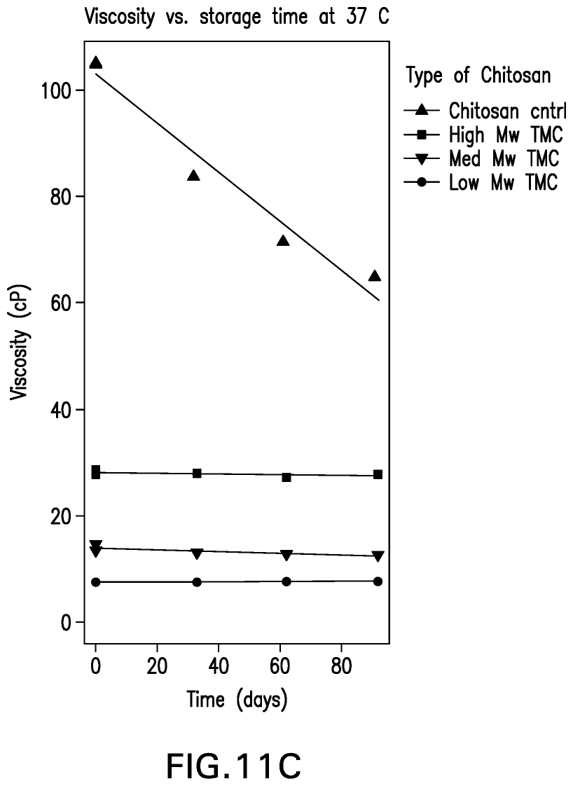

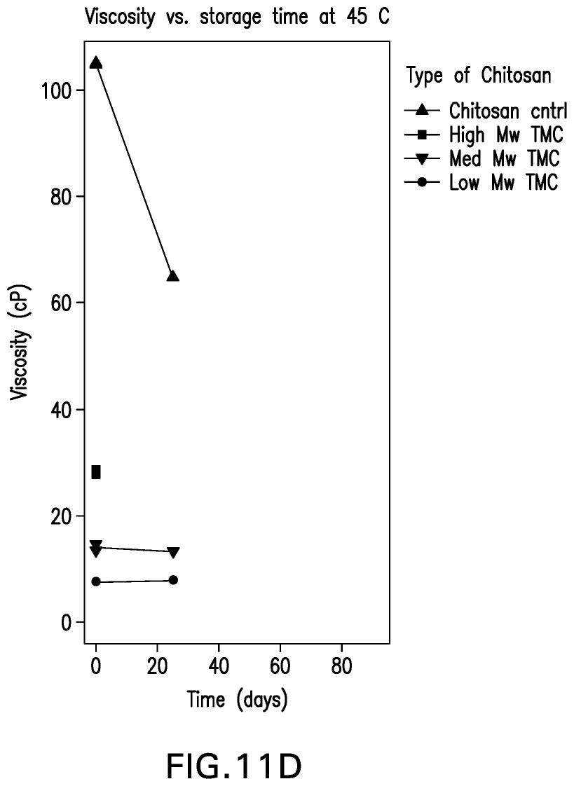

The viscosity of the undiluted samples (at approximately room temperature−instrument incubator was set at 20° C.) was measured using a microVisc capillary viscometer (RheoSense, California, USA). As seen in FIG. 11, despite being significantly larger in average molecular weight, and despite being higher concentration (for the medium and high molecular weight trimethyl chitosan), the trimethyl chitosan samples were much less viscous than the chitosan sample. Furthermore, the viscosity of the trimethyl chitosan samples remained essentially unchanged at different temperatures, and as a function of storage time; however, the viscosity of the chitosan sample changed significantly as a function of temperature and storage time.

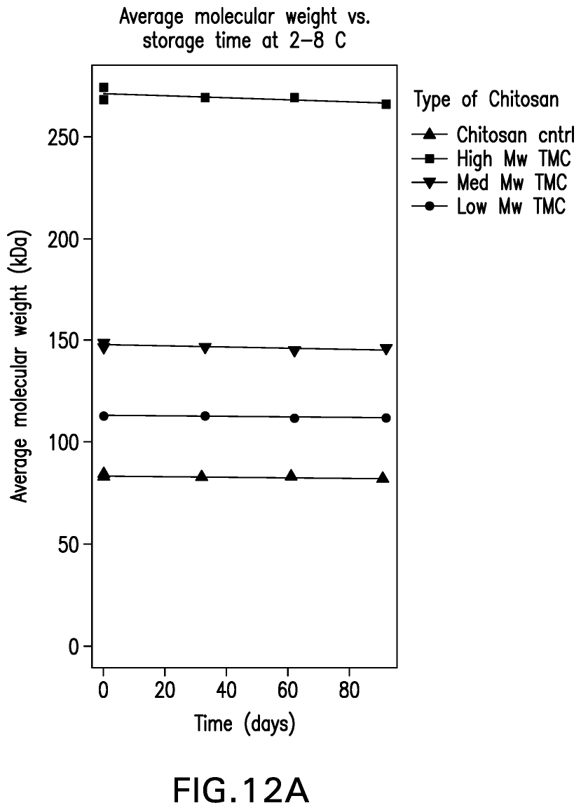

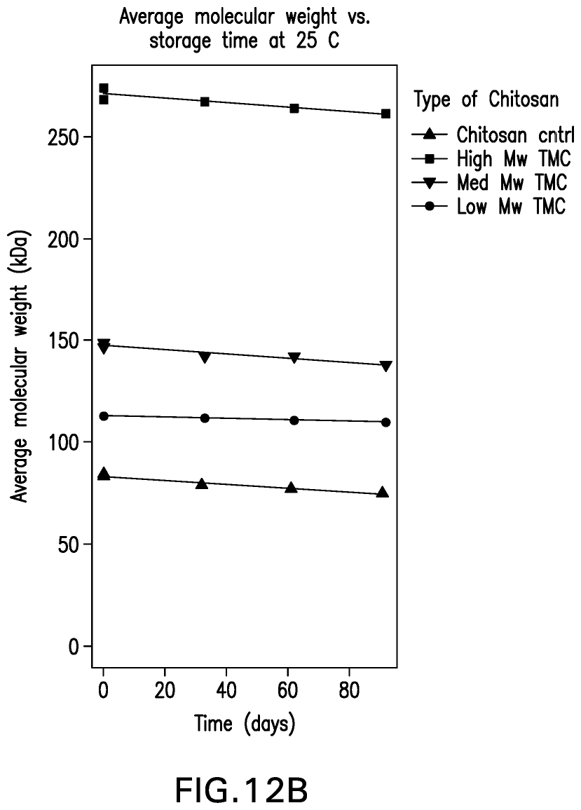

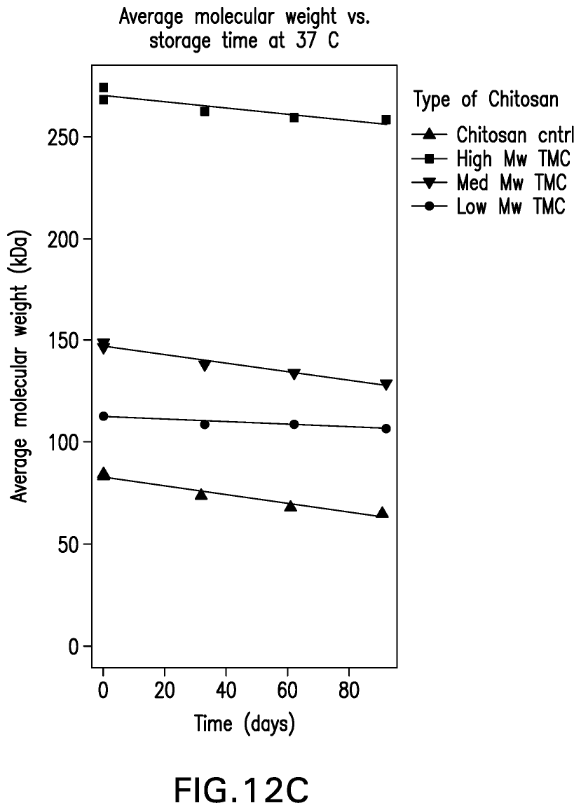

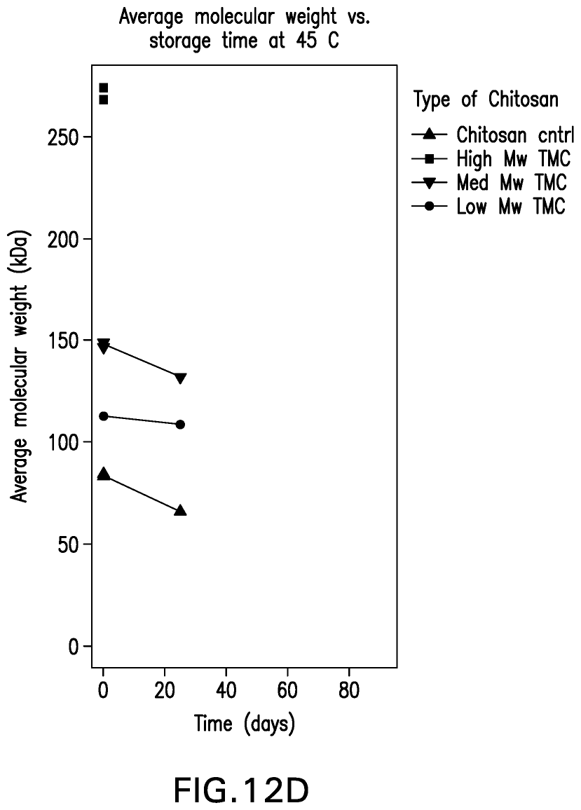

As shown in FIGS. 12A-12D, the stability of the molecular weight of chitosan in this study appears much more consistent with the stability of the molecular weight for the three trimethyl chitosan samples. Modeling the modest decline in average molecular weight over time with a simple line, some estimates of change in average molecular weight kDa per day at the different storage temperatures is shown in Table VI. As these molecules retained adjuvant activity when they had an average molecular weight at least as small as about 15 kDa, the time to crossing that size threshold with these modest rates of decay is quite significant. This could especially be the case for large average molecular weight trimethyl chitosan samples, which could advantageously possess both low viscosity and a long shelf life, retaining adjuvant activity even throughout a process of average molecular weight decrease over time.

TABLE VI
Estimates of the change in molecular weight ( kDa) per day
for chitosan and trimethyl chitosan (as formulated and
vialed in the study) at different storage temperatures.

| | 2-8° C. | 25° C. | 37° C. |
|---|---|---|---|
| Chitosan control | −0.014 | −0.101 | −0.225 |
| Low MW trimethyl chitosan | −0.011 | −0.032 | −0.07 |
| Medium MW trimethyl chitosan | −0.03 | −0.111 | −0.22 |
| High MW trimethyl chitosan | −0.049 | −0.111 | −0.167 |

The surprising low viscosity of trimethyl chitosan, and its stability in terms of both viscosity and molecular weight, even in a formulation as simple as water alone, are useful and beneficial properties for the use of soluble trimethyl chitosan in pharmaceutical formulations.

### Example 9: Adjuvant Activity of Trimethyl Chitosan Comparable to Chitosan in a Mouse Immunogenicity Study with RSV Antigen

A mouse immunogenicity study was conducted to compare the adjuvant activity of the chitosan and trimethyl chitosan samples described in Example 8 (the “non-thermally-stressed” T=0 samples). An RSV subunit pre-F protein vaccine. DS-Cav1 (“RSV Vaccine”) was made similar to what has been previously described by McLellan J S, Chen M, Joyce M G, Sastry M, Stewart-Jones G B, Yang Y, et al., Structure-based design of a fusion glycoprotein vaccine for respiratory syncytial virus. Science 2013 Nov. 1; 342(6158):592-8. The DS-Cav1 and variant RSV F sequences were codon optimized for mammalian codon usage (Life Technologies), cloned into an expression vector, and transiently transfected into Expi293 suspension cells (Life Technologies). Cell culture supernatants were harvested day 3 to 7 post-plasmid transfection and evaluated in western blot and ELISA assays described below. To obtain purified RSV F proteins, cell culture supernatants were purified using a modified method based on the procedure previously described by McLellan et al. Briefly, his-tagged proteins were purified using Ni-Sepharose chromatography (GE Healthcare). Tags were removed by overnight digestion with thrombin. Digestion was performed during dialysis to reduce imidazole concentration. To remove co-eluting contaminants and uncleaved F protein, samples were subjected to a second Ni-Sepharose chromatography step. F proteins were further purified by gel filtration chromatography (Superdex 200, GE Healthcare) and were stored in a buffer of 50 mM HEPES pH 7.5, 300 mM NaCl.

The RSV Vaccine was then combined with a chitosan or one of the three trimethyl chitosans. Each of the adjuvants (chitosan, low average MW trimethyl chitosan, medium average MW trimethyl chitosan, or high average MW trimethyl chitosan) were tested at both a low and a high dose level. The study involved 9 groups with 10 mice per group, and each mouse received 2 vaccinations, given three weeks apart (second dose on or about day 21). To assess immunogenicity and adjuvant activity, the total IgG ELISA titers and the serum neutralizing antibody titers were measured at week 5 (on or about day 35).

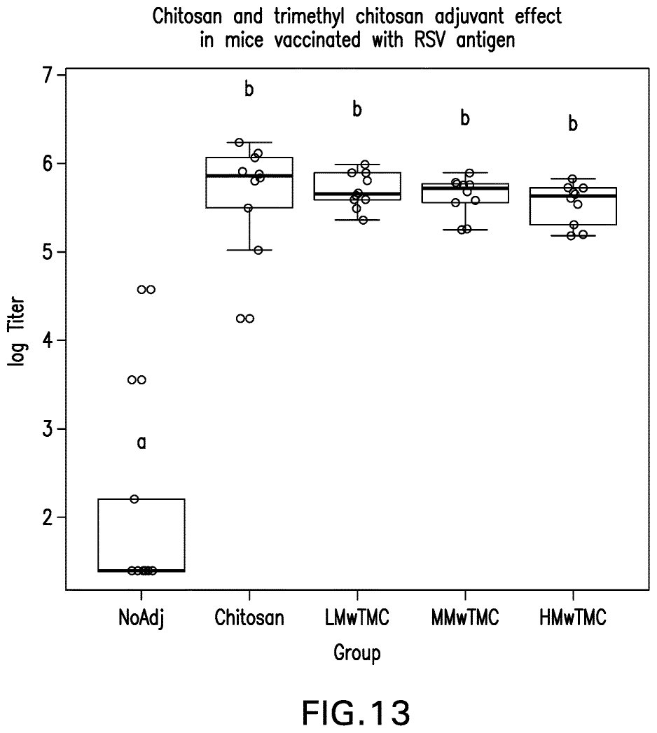

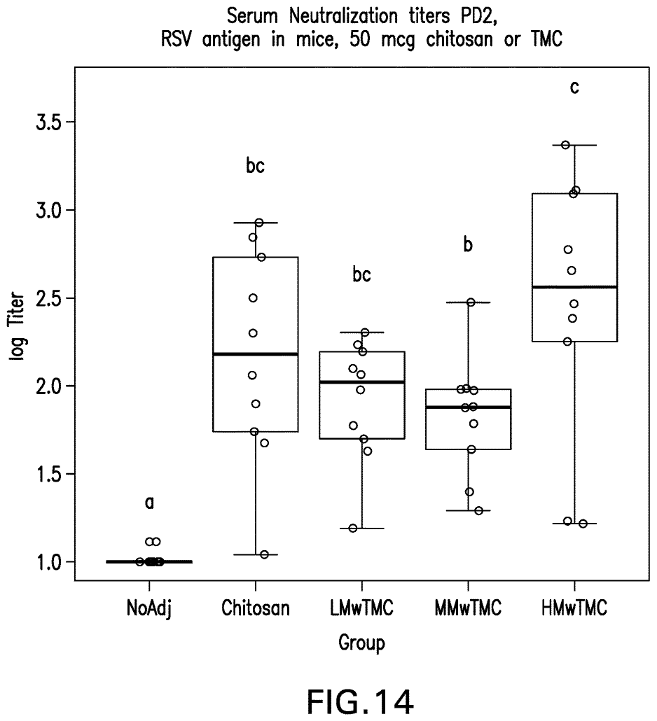

FIG. 13 shows the immune responses for the higher adjuvant dose, demonstrating a significant and comparable adjuvant activity in mice with the RSV antigen. FIG. 14 shows the serum neutralizing antibody titers for the higher dose, supporting significant and comparable adjuvant activity as well. It was found that the trimethyl chitosan provided viscosity advantages, as well as retaining its high adjuvant activity.

1. A pharmaceutical composition comprising an active biological ingredient (ABI), a chitosan or a chitosan derivative, and a pharmaceutically acceptable carrier.

2. The pharmaceutical composition of claim 1, wherein the pharmaceutical composition is made by mixing a vaccine and the chitosan or chitosan derivative; wherein the vaccine comprises the ABI and a pharmaceutically acceptable carrier.

3. The pharmaceutical composition of claim 1, wherein the composition comprises a chitosan that is an acid soluble chitosan.

4. The pharmaceutical composition of claim 1, wherein the composition comprises a chitosan that is a water-soluble chitosan.

5. The pharmaceutical composition of claim 1, wherein the composition comprises a chitosan that has a molecular weight of 5 kDa to 100 kDa.

6. The pharmaceutical composition of claim 1, wherein the composition comprises a chitosan that has a viscosity of about 1 cP to about 125 cP at 20° C., when measured with a viscometer at 20° C. at a standard concentration.

7. The pharmaceutical composition of claim 1, wherein the composition comprises a chitosan derivative that is a trimethyl chitosan.

8. The pharmaceutical composition of claim 1, wherein the composition comprises a chitosan derivative that has a molecular weight greater than 100 kDa.

9. The pharmaceutical composition of claim 1, wherein the composition comprises a chitosan derivative that has a viscosity of about 5 cP to about 30 cP at 20° C., when measured with a viscometer at 20° C. at a standard concentration.

10. The pharmaceutical composition of claim 7, wherein the pharmaceutical composition comprises trimethyl chitosan having an average molecular weight of between about 225 kDa and about 275 kDa.

11. The pharmaceutical composition of claim 7, wherein the pharmaceutical composition comprises trimethyl chitosan having an average molecular weight of between about 125 kDa and about 160 kDa.

12. The pharmaceutical composition of claim 7, wherein the pharmaceutical composition comprises trimethyl chitosan having an average molecular weight of between about 100 kDa and about 150 kDa.

13. The pharmaceutical composition of claim 7, wherein the pharmaceutical composition comprises trimethyl chitosan having an average molecular weight of between about 225 kDa and about 275 kDa and a degree of quaternization of about 30-70%.

14. The pharmaceutical composition of claim 7, wherein the pharmaceutical composition comprises trimethyl chitosan having an average molecular weight of between about 125 kDa and about 160 kDa and a degree of quaternization of about 40-60%.

15. The pharmaceutical composition of claim 7, wherein the pharmaceutical composition comprises trimethyl chitosan having an average molecular weight of between about 100 kDa and 150 kDa and a degree of quaternization of about 50% or greater.

16. The pharmaceutical composition of claim 1, wherein the pharmaceutical composition further comprises a salt.

17. The pharmaceutical composition of claim 1, wherein the pharmaceutical composition further comprises a buffer present in an amount of about 1 mMol to about 100 mMol, wherein the buffer is selected from the group consisting of: acetic acid, histidine, citrate, Bis-Tris, HEPES, phosphate, MES, sodium chloride, and combinations thereof.

18. The pharmaceutical composition of claim 1, wherein the pharmaceutical composition further comprises a tonicity modifier present in an amount of about 10 mMol to about 50 mMol, wherein the tonicity modifier is selected from the group consisting of: sodium chloride, potassium chloride, sucrose, trehalose and combinations thereof.

19. The pharmaceutical composition of claim 1, wherein the pharmaceutical composition further comprises a detergent present in an amount of about 0.001% weight per volume (w/v) to about 0.2% (w/v), wherein the detergent is selected from the group consisting of Polysorbate 80, Polysorbate 20, Poloxamer 188, and combinations thereof.

20. The pharmaceutical composition of claim 1, wherein the pharmaceutical composition further comprises an aluminum adjuvant.

21. The pharmaceutical composition of claim 1, wherein the ABI comprises a respiratory syncytial virus (RSV) antigen.

22. The pharmaceutical composition of claim 1, wherein the ABI comprises a recombinant RSV F trimer, wherein the RSV F trimer includes DS-Cav1 substitutions.

23. The pharmaceutical composition of claim 1, wherein the ABI comprises virus-like particles (VLPs) of human papillomavirus.

24. The pharmaceutical composition of claim 23, wherein composition comprises HPV VLPs of at least one HPV type selected from the group consisting of HPV type 6, 11, 16, 18, 26, 31, 33, 35, 39, 45, 51, 52, 53, 55, 56, 58, 59, 66, 68, 69, 70, 73, and 82.

25-28. (canceled)

29. The pharmaceutical composition of claim 23, wherein the HPV VLPs comprise recombinant HPV L1 or recombinant HPV L1+L2 protein.

30. The pharmaceutical composition of claim 24, wherein the HPV VLPs of each of the at least one HPV types are present in a concentration of about 10 μg to about 300 μg per 0.5 mL of the pharmaceutical composition, and wherein the total HPV VLP concentration is between 10 μg and 2000 μg per 0.5 mL of the pharmaceutical composition.

31. (canceled)

32. A method of inducing an immune response to an antigen in a human patient comprising administering to the patient the pharmaceutical composition of claim 1.

33. A method of inducing an immune response to an antigen in a human patient comprising co-administering to the patient (a) a pharmaceutical composition comprising an ABI and (b) a chitosan or chitosan derivative.

34. A method of preventing infection of a human patient with a pathogen comprising administering to the patient the pharmaceutical composition of claim 1, wherein the ABI comprises an antigen that is capable of eliciting an immune response directed against the pathogen.

35. A kit comprising:
(a) a vaccine comprising an active biological ingredient (ABI); and
(b) a chitosan or a chitosan derivative.

36. The kit of claim 35, further comprising instructions for administering to a human patient the vaccine and the chitosan or chitosan derivative.

37. A method of inducing a neutralizing immune response against an antigen in a patient in need thereof comprising:
administering to the patient a pharmaceutical composition comprising:
a chitosan or chitosan derivative, and
an active biological ingredient (ABI) comprising an antigen,
whereby the administration of the pharmaceutical composition induces a neutralizing immune response against the antigen in the patient.
